(19) United States
(12) Patent Application Publication
(10) Pub. No.: US 2012/0184642 A1
(43) Pub. Date: Jul. 19, 2012

(54) MULTIMODAL VISIBLE POLYMER EMBOLIZATION MATERIAL

(76) Inventors: Soenke Bartling, Heidelberg (DE); Shlomo Margel, Rehovot (IL); Hagit Aviv, Givart Shmuel (IL)

(21) Appl. No.: 13/382,352

(22) PCT Filed: Jul. 6, 2010
(86) PCT No.: PCT/EP2010/059631

§ 371 Date: Mar. 23, 2012

(30) Foreign Application Priority Data
Jul. 7, 2009 (DE) 102009032189.6

(51) Int. Cl. A61L 31/04; A61L 31/06; A61L 31/18

(52) U.S. Cl. 523/113

(57) ABSTRACT

The present invention relates to embolization material for therapeutic use, wherein said material is visible via more than one imaging technique.

The present invention relates to embolization material for therapeutic use, wherein said material is visible via more than one imaging technique.

Embolization therapy is a common therapeutical concept to treat pathological alterations inside the human body. Generally, vessels are blocked by an intravascular application of a material. Various substances can be introduced into the circulation (bloodstream) to occlude vessels, for example to arrest or prevent hemorrhaging, to devitalize a structure, tumor, or organ by occluding its blood supply; or to reduce blood flow to an arteriovenous malformation, or other vascular malformation. For this purpose different materials have been tested which are termed embolization materials or embolization agents synonymously in the following.

Generally, embolization material or vascular embolization agents are particles (non-spherical or microspherical) or fluids (glues, gels, sclerosing agents and viscous emulsions) that can be released into the bloodstream through a catheter or needle to mechanically and/or biologically occlude the target vessels, either permanently or temporarily. Commonly, these materials are available as solids, liquids or suspensions. In principle, a selection of the embolization agent based on the size and the calibre of the target vessels ensures that the occlusion is confined to the desired site. Basically, particles cause mechanical occlusion, whereas glues and gelling solutions solidify at the target, and e.g. acetic acid, ethanol, and various sclerosing agents modify the vessel wall and contents, leading to the development of a clot that occludes the vessel (Loffroy et al., “Endovascular Therapeutic Embolisation An Overview of Occluding Agents and their Effects on Embolised Tissues”, Current Vasc Pharmacology, 2009, 7, 1-14).

Common treatment of vascular defects, e.g. intracranial aneurysms, is performed using neurosurgical clipping. A viable alternative for treatment of such conditions is endovascular embolization with platinum coils. High numbers of patients having a recurrence amenable to retreatment because of thrombus recanalization, aneurysm regrowth, or embolic mass compaction led to development and clinical use of embolic devices combining platinum coils with expandable hydrogels or degradable polymers to reduce the retreatment rate. For example a dried hydrogel is placed over a platinum coil, or degradable polymers such as copolymers of glycolic acid and lactic acid are placed over and/or inside a platinum coil. Besides, other materials, e.g. hydrogel filaments are currently used as implants for endovascular embolization such as poly(ethylene), poly(ethylene glycol) diacrylate with 2,4,6-triiodophenyl penta-4-enoate (PEG-I), poly(ethylene glycol) diacrylamide with barium sulfate (PEG-B), poly(propylene glycol) diacrylate with barium sulfate (PPG-B) (Constant et al., “Preparation, Characterization, and Evaluation of Radiopaque Hydrogel Filaments for Endovascular Embolization”, J Biomedical Mat Research Part B, Appl Biomaterials, 2008, 306-313). Nevertheless, currently available embolization devices are either not visible (e.g. not radio-opaque, or magnetic) by medical imaging techniques or visible only via CT but due to the metallic nature of platinum leading to imaging artifacts.

Embolization is frequently conducted under control of medical imaging techniques including inter alia projectional or plain radiography (X-ray based angiography), magnetic resonance angiography (MRA) based on magnetic resonance imaging (MRI) and other radiography methods. Embolization is carried out either trans-arterial via micro catheter or via direct puncture, whereby the embolization agent (e.g. occlusion emulsion) is injected via puncture needle into the target region. DE 102 61 694 describes injection of a liquid embolization agent containing a protein emulsion (Zein) and ethanol.

Common imaging techniques in radiology are angiography, X-ray computed tomography (CT), radiography, magnetic resonance imaging (MRI), ultrasonography (US), nuclear medical techniques such as single photon emission computed tomography (SPECT) and positron emission tomography (PET), optical techniques, techniques enabling localization via radio waves, and magnetic particle imaging technique. Embolization agents visible via radiology techniques enable their detection, localization, control of therapy by the aforementioned techniques, and their display whilst application regarding the human body and pathological alterations.

Currently, clinical embolization materials are not visible by imaging techniques (Siskin et al., “Embolic Agents Used for Uterine Fibroid Embolization”, American Journal of Roentgenology, 2000, 767-773). However, it is accepted that directly visible embolization material provides advantages over non-visible embolization materials (Mottu et al., “Iodine-containing cellulose mixed esters as radiopaque polymers for direct embolization of cerebral aneurysms and arteriovenous malformations”, Biomaterials, 2002, 23, 121-131; Siskin et al., loc. cit.; Sharma et al., “Development of “imageable” beads for transcatheter embolotherapy”, J Vasc Intery Radio, 2010, 21(6), 865-76).

An embolization material that is directly visible by an imaging modality provides advantages to control the application of the embolization material, to verify and document the therapy success and might provide methods to detect misplacement of embolization material.

Tumor embolization is currently mostly performed under X-ray control for application catheter placement, treatment planning as well as treatment control (Lubienski et al., “Update Chemoperfusion and -embolisation”, Der Radiologe, 2007, vol. 47, 1097-106).

It was proposed to switch to an MRI environment for embolization therapy, because this would reduce or eliminate the necessary radiation dose, and would enable three-dimensional therapy control. As a consequence this would widen the potential range of therapies and therefore increase the spectrum of potential patients. Especially young women with uterus fibroids could now undergo embolization treatment without potential harm to the very radiation sensitive ovaries (Levy, “Modern management of uterine fibroids”, Acta Obstet Gynecol Scand, 2008, 87(8), 812-23).

Various embolization materials exist being visible via one radiology technique (X-ray computed tomography (CT), radiography). There are also embolization materials being visible via other imaging techniques, see for example DE 102 61 694 (Zein-emulsion with radiocontrast agent); DE 09414868 U1 (synthetic particle with Iodine), US 2005/0095428 (polymer with Ni—Ti-alloy), and WO 2001/66016 (gas containing embolization agents).

However, no embolization materials have been described which are well visible in more than one imaging technique. Thus, there is currently a dependency on one imaging technique for controlling the application of embolization material and for controlling therapy. Up to now, it is not possible to combine the advantages of different imaging techniques.

Projectional radiography is a currently used environment to carry out embolization. Physicians have the most experience here. A disadvantage of this technique is that their application is connected with ionizing radiation which may possibly cause cancer.

In future, it is envisaged to increasingly conduct embolization therapy in healthy patients having benign tumors, which otherwise have to be surgically treated. Here, it is often important to avoid ionizing radiation. Embolization can for example be carried out under control of MRI. Embolization materials only visible by MRI averts control via X-ray computed tomography (CT) or projectional radiography.

Current embolization therapy is mainly done by projective imaging enabling assessment of embolization success in two dimensions only. In future, rotational angiography, cone-beam CT, dyna-CT, MRI, ultrasonography, or magnetic particle imaging will enable availability of three-dimensional imaging.

Another disadvantage of currently known embolization agents lies in the control of therapy. Control of therapy should enable visualization e.g. which regions of tumor vessels are successfully occluded. Embolization and control of therapy is often carried out using one imaging technique, and thus, is restricted to it. So, there is no possibility to visualize embolizations and control of therapy via a second or a third imaging technique in order to combine their advantages.

If embolization material gets into regions of the human body or vessels not destined to be there, e.g. healthy regions, this process is called misplacement of embolization material. Currently available embolization material can only be visualized via one imaging technique, and thus, misplaced embolization material needs to be detected using this one imaging technique. To date no combination of the advantages of different imaging techniques is possible. CT for example requires radio-opaque embolization materials and is the superior mode of action to visualize lung regions compared to MRI. However, MRI visualization requires certain characteristics of embolization material. MRI is the superior mode of action for visualization of soft tissues structures compared to CT. A combination of both techniques to enable detection in the whole human body is not feasible according to the actual prior art because embolization material is only visible either via CT or via MRI.

Current embolization materials are only visible by one imaging technique at a time, and thus, can not be imaged by more than one imaging technique. This restricts the therapy control, application and application control of a certain embolization material.

Changing of the imaging environment during intervention comes along with a loss of the ability to track the embolization material and perform embolization treatment evaluation. Thus, there is a need for embolization material visible via different imaging techniques.

Furthermore, currently multimodally interventional imaging systems so called hybrid systems are going to be developed in interventional radiology. (Fahrig et al., “A truly hybrid interventional MR/X-ray system: feasibility demonstration”, J Magn Reson Imaging, 2001, 13(2), 294-300; Kee et al., “MR-guided transjugular intrahepatic portosystemic shunt creation with use of a hybrid radiography/MR system”, J Vasc Intery Radiol, 2005, 227-34; Wilson et al., “Experimental Renal Artery Embolization in a Combined MR Imaging/Angiographic Unit”, J Vasc Intery Radiol, 2003, 14, 1169-1175). In such systems, several partly complementary imaging techniques are integrated into one work space (Fahrig et al., loc. cit.; Kee et al., loc. cit.). But, the currently known embolization materials are visible only via one of those imaging techniques. Thus, the inherent advantages of such hybrid systems, the advantages of combining several imaging techniques can not be used for detection of embolization material.

Thus, one technical problem underlying the present invention is seen as the provision of materials and methods for enabling the visualization of embolization, of the application of the embolization therapy and of the control of therapy and treatment using more than one imaging technique in order to combine the specific advantages of the respective imaging techniques.

The problem is solved by the embodiments of the present invention described in the claims and the specification herein below. Specifically, the problem is solved by the provision of embolization material for therapeutic applications visible via more than one imaging technique.

The present invention relates to embolization material for therapeutic use, wherein said material is visible via more than one imaging technique.

In one aspect of the invention, the embolization material of the present invention comprises at least one polymer component and at least one inorganic component, and said embolization material is visible with high contrast via more than one imaging technique, in particular by two different techniques, by three different techniques or more.

The term “high contrast” as used in accordance with the present invention, relates to contrast enhanced by contrast agents in one respective imaging technique in the clinical practice. Generally, contrast is the difference in blackness, whiteness, or other colorness, between two adjacent tones. High contrast is further characterized as an accurate portrayal of the structures under examination in good positioning with the minimum of geometric distortion, easy perception of the relevant structures in detail, and without or very little misleading artifacts. Furthermore, “high contrast” relates to the contrast which enables clarification of diagnostic problems via at least two different imaging techniques. Moreover, contrast as used herein refers to the embolization material of the present invention visible in at least two imaging techniques already in marginal density of said embolization material.

In another aspect, the embolization material of the present invention is visible via the following imaging techniques (at the same time):
a) X-ray computed tomography (CT)/projectional radiography and magnetic resonance imaging (MRI),
b) X-ray computed tomography (CT)/projectional radiography and ultrasonography (US),
c) X-ray computed tomography (CT)/projectional radiography and single photon emission computed tomography (SPECT),
d) X-ray computed tomography (CT)/projectional radiography and positron emission tomography (PET),
e) magnetic resonance imaging (MRI) and ultrasonography (US),
f) magnetic resonance imaging (MRI) and single photon emission computed tomography (SPECT),
g) magnetic resonance imaging (MRI) and positron emission tomography (PET),
h) X-ray computed tomography (CT)/projectional radiography and magnetic particle imaging, or
i) a combination of two or more of said imaging techniques.

In an aspect of the present invention the embolization material is visible via three imaging techniques at the same time.

The term “at the same time” as used in accordance with the present invention relates to the embolization material of the present invention being visible via one imaging technique as well as via another imaging technique either at the same moment or in close sequence (often also via even more imaging techniques).

Generally, embolization material as used in accordance with the present invention relates to material consisting of a mixture of different components. The embolization material frequently contains at least one polymer component and at least one inorganic component as described in more detail herein below.

In an aspect, the embolization material of present invention comprises embolization material, wherein the at least one polymer component is selected from the group of polyacrylate, polymethacrylate, polyacrylamide, polymethacrylamide, acrylate polymer, polyamide, polysiloxane, polyester, polyurethane, polyvinyl ether, polyvinyl ester, copolymers comprising as monomers a (meth)acrylic-derivative and/or a meth(acrylamide)-derivative carrying a cleavable iodine substituted side group, or mixtures thereof.

In another aspect, of the embolization material of the present invention, the at least one polymer component comprises a copolymer of glycidyl-methacrylate and a (meth)acrylic-derivative carrying a cleavable iodine substituted aromatic side group.

In a further aspect, the embolization material of present invention comprises at least one polymer or copolymer component selected from the group of polyacrylate, polymethacrylate, polyacrylamide, polymethacrylamide, acrylate polymer, polyamide, polysiloxane, polyester, polyurethane, polyvinyl ether, polyvinyl ester, copolymer of 2-methacryloyloxyethyl (2,3,5-triiodobenzoate) and methyl-methacrylate, or mixtures thereof.

In another aspect, in accordance with the present invention, the polymer is selected from the group consisting of polyacrylate and polymethacrylate. In a further aspect of the embolization material of the present invention, the at least one polymer component comprises a copolymer of 2-methacryloyloxyethyl (2,3,5-triiodobenzoate) and methyl-methacrylate. Also in accordance with the present invention, the polymer component can further be selected from biodegradable polymeric spheres, or polyesters of tetraiodophenolphthalein.

The term “polymer”, as used herein, includes homo-polymers and copolymers. In the case of a co-polymer, said co-polymer can preferably be the polymerization product of two or more iodine substituted monomers, or alternatively, the polymerization product of at least one iodine substituted monomer with at least one bi-functional monomer that contains, in addition to its polymerizable functionality, a second reactive chemical group, e.g., glycidol methacrylate.

The embolization material of the invention often comprises as at least one polymer component a copolymer of (meth)acrylic and meth(acrylamide) monomers carrying cleavable iodine substituted side groups.

According to the present invention, the polymer component can further contain monomers, e.g. vinylic monomers, e.g. hydroxyethyl methacrylate (HEMA), acryloyl chloride, methacryloyl chloride, and/or glycidyl methacrylate. Especially preferred are (meth)acrylic and meth(acrylamide) monomers which carry cleavable radio-opaque element (e.g. iodine) substituted side groups.

In an aspect, of the embolization material of the present invention, the at least one inorganic component comprises a radio-opaque element selected from the group of calcium, iron, iodine, xenon, barium, ytterbium, silver, gold, bismuth, cesium, thorium, or tungsten, and a magnetic resonance imaging (MRI) visible component selected from the group iron oxides, gadolinium, manganese, or perfluorocarbons.

In another aspect, further radio-opaque elements selected from Iodine with ionic or nonionic monomers (e.g. Diatrizoate, Iohexyl), dimers (e.g. Ioxaglate, Iodixanol), or polymers, Barium, electrondense heavy metals, rare earth elements, with chelates e.g. EDTA, DOTA are also in accordance with the present invention.

Moreover, further components visible via MRI selected from gadolinium based contrast agents, e.g. gadodiamide, gadobenic acid, gadopentetic acid, gadoteridol, gadofosveset, gadoversetamide), gadoxetic acid, gadobutrol, gadocoletic acid, gadodenterate, gadomelitol, gadopenamide, gadoteric acid, manganese based contrast agents (e.g. Mn-DPDP), Ferumoxsil, Ferristene, or diamagnetic, ferromagnetic, paramagnetic substances in Small- or Ultra Small Super Paramagnetic Iron Oxid (SPIOs/USPIOs) with or without chelates are also in accordance with the present invention.

In another aspect, the embolization material of the present invention comprises a radio-opaque element, and a magnetic resonance imaging (MRI) visible component, and additionally components enabling detection via ultrasonography (US) selected from the group of gas aggregates or gas bubbles, microbubbles, microspheres of human albumin, microparticles of galactose, perflenapent, microspheres of phospholipids, and/or sulfur hexafluoride. In an aspect, said components enabling detection via ultrasonography are coated or incorporated into the embolization material of the present invention.

Moreover, in accordance with the present invention, the embolization material can comprise further microspheres. Microspheres as used herein, consist of various materials e.g. glass, silicone, polyvinyl-alcohol-hydrogels (PVA), or micellar components, e.g. micellar block-copolymers, or liposomes. Further, microspheres as used in accordance with the present invention can be loaded with Lipiodol and/or gadolinium. Also contemplated, in accordance with the present invention, are microspheres of human albumin, microspheres of phospholipids, and/or sulfur hexafluoride.

Moreover, in a further aspect, the embolization material of the present invention is visible via PET. Thus, in an aspect, the embolization material comprises positron emitters, e.g. zirconium-89, iodine-124, radionuclides, e.g. technetium-99m, (^{99m}Tc), molybdenum-99, positrons, beta-ray-emitters, e.g. fluorine-18 (F-18), carbon-11 (C-11), nitrogen-13 (N-13) and oxygen-15 (0-15).

In even another aspect, the embolization material of the present invention is visible via SPECT. Thus, in accordance with the present invention the embolization material comprises gamma-ray emitters, e.g. technetium-99m, iodine-123, indium-111.

The term “iron oxide” as used in accordance with the present invention relates to Fe₃O₄, Fe₂O₃, or FeO.

In an even further aspect, the embolization material of the present invention comprises an X-ray visible, iodine containing core, and a MRI visible, ultra small paramagnetic iron oxide based coating, e.g. Fe₃O₄, Fe₂O₃, and wherein said material is selected from magnetic iron oxide/Poly((2-methacryloyloxyethyl-(2,3,5-triiodobenzoate))-(glycidyl-methacrylate)) particles.

In even another aspect, the embolization material of the present invention comprises a MRI visible, ultra small paramagnetic iron oxide based core and an X-ray visible, iodine containing coating, e.g. the aforementioned polymer component, or a mixture of both materials.

In an aspect, the embolization material of present invention exhibits different particle sizes ranging from 30 μm to 900 μm and is detectable in a first imaging technique displaying good localization and at the same time in a second very sensitive imaging technique. In accordance with the present invention, said first imaging technique is selected from X-ray computed tomography (CT)/projectional radiography, or magnetic resonance imaging (MRI). Further, in accordance with the present invention, said second imaging technique is selected from ultrasonography (US) and nuclear medical imaging techniques. In an even further aspect, the embolization material of present invention exhibits particle sizes ranging from 40 μm to 200 μm.

Moreover, the present invention relates to a kit of at least two parts for the preparation of embolization material of the invention, the kit comprising as one part at least one polymer component and as second part at least one inorganic component.

Furthermore, the present invention also relates to a method for the preparation of the embolization material of the invention comprising the steps of:
a) synthesizing the at least one polymer component,
b) synthesizing the at least one inorganic component, and
c) optionally synthesizing a component detectable via ultrasonography, and
d) combining the at least one polymer component of step a with the at least one inorganic component of step b, and optionally with the component of step c, and thus, obtaining the embolization material of any one of claims 1 to 13.

According to the present invention embolization material can be used for different therapeutic applications, e.g. for occlusion of vessels inside the human or animal body. Specifically, the embolization material of the present invention can be used for occlusion, e.g. occlusion of specific vessels, occlusion of bile ducts, or fistulae, and/or the treatment of aneurysms. This is achieved by adjusting size, stability, structure and/or (inflammation-) stimuli triggering features of the embolization material. Size can be variably adjusted in order to directly target different vessel regions (e.g. big or small tumor vessels), or other targets. The embolization material can exhibit different moieties, coating, charging, or cover to target different vessels or other targets as described above.

Moreover, the embolization material of the present invention enable using special characteristics of different imaging techniques, e.g. to combine partially complementary characteristics for quantification, sensible detection, shunt prevention, assessment of particle distribution, and/or real-time imaging.

Also contemplated by the present invention is the use of the embolization material of present invention together with chemotherapeutics, internal radiation sources, targeted moieties, and/or activatable probes.

The present invention also relates to the use of embolization material of the present invention detectable via ultrasonography to trace real-time shunting, and/or enable sensible detection of the particles within tumor or shunting vessels. Moreover, the characteristics of embolization material containing US detectable components can be changed by destroying of gas aggregates or gas bubbles, thus even broadening the spectrum of their uses.

Further, and also according to the present invention, the embolization material can additionally contain active ingredients and/or excipients. Active ingredients could for example be anti-thrombolytic agents such as heparin, derivatives of heparin, or urokinase. Also anti-proliferating agents such as enoxaparin, angiopeptin, hirudin or acetylsalicylic acid, and anti-inflammatory agents such as dexamethasone, corticosteroids, budesonide, sulfasalazine or mesalamine can be used. Typical oncologic active ingredients such as cisplatin, paclitaxel, vinblastine, angiostatin, or fluorouracil can also be used in the composition. The embolization material can contain as additional component an anesthetic agent such as lidocaine, bupivacaine, or ropivacaine. Common anticoagulants can also be contained.

The term “multimodality embolization material” as used in the present invention refers to embolization material visible via more than one, in particular via two, three, or more imaging techniques.

The embolization material due to its composition is visible via more than one medical imaging technique. Thus, control of therapy while application and thereafter can be carried out via more than one imaging technique. Because medical imaging techniques differ from one another and are partly complementary, a combination of several imaging techniques can unite the advantages of each technique.

The present invention relates to the application of embolization materials that can be visualized via several imaging techniques. Thus, the advantages of the imaging techniques can be combined. Long-term control of therapy can also be carried out using several imaging techniques. For example, definite embolized regions of tumors can be distinguished from non-embolized regions. This can be carried out using different imaging techniques. For instance benign tumors, such as uterine myoma can be embolized in an X-ray environment (projectional radiography), but therapy control can be done in a low radiation environment via MRI. Furthermore, several imaging techniques can be combined for detection of misplaced embolization material. For example, MRI-particles tagged with iron oxide particles such as “Ultra Small Super Paramagnetic Iron Oxid” (USPIO) (Weissleder et al., “Ultrasmall Superparamagnetic Iron Oxide: Characterization of a New Class of Contrast Agents for MR-Imaging”, Radiology, 1990, 175, 489-493) can easily be detected in soft tissues, whereas particles in the lung can be detected with good results via CT, since MRI is limited regarding good imaging quality in the lung. Moreover, considering that imaging techniques can differ in their sensitivities for different regions of the body, (e.g. MRI can be more sensitive than CT) a combination of several imaging techniques can increase overall sensitivity. Highest sensitivities for visualization can be achieved by using nuclear medical imaging technique. A combination of radio-opaque embolization material with nuclear medical tracer enables for example optimal control for application and optimal detection of misplaced embolization material. So, already smallest amounts of misplaced embolization material can be detected. This can possibly be relevant for selective internal radio-therapy (SIRT) a form of radiation therapy used to treat cancer. It is generally for selected patients with unresectable cancers, those which cannot be treated surgically, especially hepatic cell carcinoma or metastasis to the liver. The treatment involves injecting tiny microspheres of radioactive material into the arteries that supply the tumor.

Using multimodality embolization materials enables therapy control to be performed via more than one imaging technique. Since imaging techniques differ in sensitivity to image certain organs and/or disease conditions multimodality embolization materials visible in several imaging techniques may complement those for therapy control and evaluation. Furthermore, imaging techniques differ regarding invasiveness and radiation harm. Embolization materials that are visible in more than one imaging techniques provide more alternatives to use the appropriate imaging technique for therapy control. The imaging technique used for application does not necessarily have to be the most suitable for therapy control. Moreover, synergistic effects of different imaging techniques might lead to new therapy concepts (e.g. real-time monitoring of misplacement of embolization particles, size testing, etc.).

According to the present invention embolization particles are preferably visible via CT as well as via MRI. Even intraprocedural switches (planned or during complications) would not come along with a loss of the ability to image the particles for application or treatment control. Using the embolization particles provided in an aspect of the present invention, displacement of embolization material could be detected by both techniques in combination. For treatment control, both CT and MRI can be used. Weaknesses of one imaging technique can be complemented by the other.

In an established multimodality hybrid intervention system (Fahrig et al., loc. cit.; Kee et al., loc. cit.) the said embolization particles are beneficial because application can be monitored using the X-ray component, while therapy control as well as monitoring can be performed using the MRI component. Furthermore, for therapy control both components X-ray CT (lung) and MRI (all other body parts) can be used synergistic. Thus, control can be carried out using both methods during examination. This means a change of imaging technique is not required.

The term “projectional or plain (film) radiography or x-ray based angiography” as used in the present invention, relates to the branch of medicine utilizing X-rays as imaging technique. Radiographs (or roentgenographs) are produced by the transmission of X-rays through a patient to a capture device then converted into an image for diagnosis. The original and still common imaging produces silver impregnated films. In Film-Screen radiography an X-ray tube generates a beam of x-rays which is aimed at the patient. The X-rays which pass through the patient are filtered to reduce scatter and noise and then strike an undeveloped film, held tight to a screen of light emitting phosphors in a light-tight cassette. The film is then developed chemically and an image appears on the film. Now replacing Film-Screen radiography is Digital Radiography, DR, in which X-rays strike a plate of sensors which then converts the signals generated into digital information and an image on computer screen. Plain radiography was the only imaging modality available during the first 50 years of radiology. It is still the first study ordered in evaluation of the lungs, heart and skeleton because of its wide availability, speed and relative low cost. New developments include the virtual X-ray system (virtX), invented by a team of computer scientists, trauma surgeons, and radiologists enabling trainees to make C-arm adjustments for different surgical procedures by using a simulation-based practice environment without X-ray exposure but with visual feedback through a digitally reconstructed radiograph (or DRR).

Interventional radiology as used in the present invention is the performance of generally minimally invasive medical procedures with the guidance of imaging techniques. The acquisition of medical imaging is usually carried out by the radiographer physicist or radiologic technologist.

The terms “other radiography methods” and “angiography” as used in the present invention relate to fluoroscopy and angiography as special applications of X-ray imaging, in which a fluorescent screen and image intensifier tube or flat panel detector is connected to a closed-circuit television system. This enables real-time imaging of structures in motion or augmented with a radiocontrast agent. Radiocontrast agents are administered, often swallowed or injected into the body of the patient, to delineate anatomy and functioning of the blood vessels, the genitourinary system or the gastrointestinal tract. Two radiocontrasts are presently in use. Barium (as BaSO₄) may be given orally or rectally for evaluation of the GI tract. Iodine, in multiple proprietary forms, may be given by oral, rectal, intraarterial or intravenous routes. These radiocontrast agents strongly absorb or scatter X-ray radiation, and in conjunction with the real-time imaging enable demonstration of dynamic processes, such as peristalsis in the digestive tract or blood flow in arteries and veins. Iodine contrast may also be concentrated in abnormal areas more or less than in normal tissues and make abnormalities (tumors, cysts, inflammation) more conspicuous. Additionally, in specific circumstances air can be used as a contrast agent for the gastrointestinal system and carbon dioxide can be used as a contrast agent in the venous system; in these cases, the contrast agent attenuates the X-ray radiation less than the surrounding tissues.

The term “X-ray computed tomography” (CT) as used in accordance with the present invention relates to an imaging technique using X-rays in conjunction with computing algorithms to image the body. Therefor, an X-ray generating tube opposite an X-ray detector (or detectors) in a ring shaped apparatus rotate around a patient producing a computer generated cross-sectional image (tomogram). CT is acquired in the axial plane, while coronal and sagittal images can be rendered by computer reconstruction. Radio contrast agents are often used with CT for enhanced delineation of anatomy. Although radiographs provide higher spatial resolution, CT can detect more subtle variations in attenuation of X-rays. CT exposes the patient to more ionizing radiation than a radiograph. Spiral Multi-detector CT utilizes 8, 16, 64 or more detectors during continuous motion of the patient through the radiation beam to obtain much finer detail images in a shorter exam time. With rapid administration of contrast during the CT scan these fine detail images can be reconstructed into three-dimensional (3D) images of carotid, cerebral and coronary arteries, CTA, CT angiography. CT scanning has become the test of choice in diagnosing some urgent and emergent conditions such as cerebral hemorrhage, pulmonary embolism (clots in the arteries of the lungs), aortic dissection (tearing of the aortic wall), appendicitis, diverticulitis, and obstructing kidney stones. Continuing improvements in CT technology including faster scanning times and improved resolution have dramatically increased the accuracy and usefulness of CT scanning and consequently increased utilization in medical diagnosis.

The term “magnetic resonance angiography” (MRA) relates to a branch of medicine utilizing magnetic resonance imaging (MRI) as imaging technique.

The term “magnetic resonance imaging” (MRI) as used in accordance with the present invention relates to an imaging technique using strong magnetic fields to align atomic nuclei (usually hydrogen protons) within body tissues, then uses a radio signal to disturb the axis of rotation of these nuclei and observes the radio frequency signal generated as the nuclei return to their baseline states plus all surrounding areas. The radio signals are collected by small antennae, called coils, placed near the area of interest. An advantage of MRI is its ability to produce images in axial, coronal, sagittal and multiple oblique planes with equal ease. MRI scans give the best soft tissue contrast of all the imaging modalities. With advances in scanning speed and spatial resolution, and improvements in computer 3D algorithms and hardware, MRI has become a versatile tool in radiology especially in musculoskeletal radiology and neuroradiology. Nevertheless, it is disadvantageous that the patient needs not to move for long periods of time in a noisy, cramped space while the imaging is performed. Claustrophobia severe enough to terminate the MRI exam is reported in up to 5% of patients. Recent improvements in magnet design including stronger magnetic fields (3 teslas), shortening exam times, wider, shorter magnet bores and more open magnet designs, have brought some relief for claustrophobic patients. However, in magnets of equal field strength there is often a trade-off between image quality and open design. MRI has great benefit in imaging the brain, spine, and musculoskeletal system. The modality is currently contraindicated for patients with pacemakers, cochlear implants, some indwelling medication pumps, certain types of cerebral aneurysm clips, metal fragments in the eyes and some metallic hardware due to the powerful magnetic fields and strong fluctuating radio signals the body is exposed to. Areas of potential advancement include functional imaging, cardiovascular MRI, as well as MR image guided therapy.

The term “radiography” as used in accordance with the present invention relates to the use of X-rays to cross materials to view inside objects. A heterogeneous beam of X-rays is produced by an X-ray generator and is projected toward an object. According to the density and composition of the different areas of the object a proportion of X-rays are absorbed by the object. The X-rays that pass through are then captured behind the object by a detector (film sensitive to X-rays or a digital detector) which gives a two-dimensional (2D) representation of all the structures superimposed on each other. In tomography, the X-ray source and detector move to blur out structures not in the focal plane. Computed tomography (CT scanning) is different to plain film tomography in that computer assisted reconstruction is used to generate a three-dimensional (3D) representation of the scanned object/patient.

The term “ultrasonography” (US) as used in accordance with the present invention relates to medical ultrasonography which uses ultrasound, i.e. high-frequency sound waves to visualize soft tissue structures in the body in real time. No ionizing radiation is involved, but the quality of the images obtained using ultrasound is highly dependent on the skill of the person (ultrasonographer) performing the exam. Ultrasound is also limited by its inability to image through air (lungs, bowel loops) or bone. The use of ultrasound in medical imaging has developed mostly within the last 30 years. The first ultrasound images were static and two dimensional (2D), but with modern-day ultrasonography 3D reconstructions can be observed in real-time; effectively becoming four-dimensional (4D). Because ultrasound does not utilize ionizing radiation, unlike radiography, CT scans, and nuclear medicine imaging techniques, it is generally considered safer. For this reason, this modality plays a vital role in obstetrical imaging. Fetal anatomic development can be thoroughly evaluated allowing early diagnosis of many fetal anomalies. Growth can be assessed over time, important in patients with chronic disease or gestation-induced disease, and in multiple gestations (twins, triplets etc.). Color-Flow Doppler Ultrasound measures the severity of peripheral vascular disease and is used by Cardiology for dynamic evaluation of the heart, heart valves and major vessels. Stenosis of the carotid arteries can presage cerebral infarcts (strokes). DVT in the legs can be found via ultrasound before it dislodges and travels to the lungs (pulmonary embolism), which can be fatal if left untreated. Ultrasound is useful for image-guided interventions like biopsies and drainages such as thoracentesis. Small portable ultrasound devices now replace peritoneal lavage in the triage of trauma victims by directly assessing for the presence of hemorrhage in the peritoneum and the integrity of the major viscera including the liver, spleen and kidneys. Extensive hemoperitoneum (bleeding inside the body cavity) or injury to the major organs may require emergent surgical exploration and repair.

The term “nuclear medical techniques” as used in accordance with the present invention relates to the branch of nuclear medicine imaging involving administration into the patient of radiopharmaceuticals consisting of substances labeled with radioactive tracer, and showing affinity for certain body tissues. The most commonly used tracers are technetium-99m, iodine-123, iodine-131, gallium-67 and thallium-201. The heart, lungs, thyroid, liver, gallbladder, and bones are commonly evaluated for particular conditions using these techniques. While anatomical detail is limited in these studies, nuclear medicine is useful in displaying physiological function. The excretory function of the kidneys, iodine concentrating ability of the thyroid, blood flow to heart muscle, etc. can be measured. The principal imaging device is the gamma camera which detects the radiation emitted by the tracer in the body and displays it as an image. With computer processing, the information can be displayed as axial, coronal and sagittal images (SPECT images, single-photon emission computed tomography). In the most modern devices nuclear medicine images can be fused with a CT scan taken quasi-simultaneously so that the physiological information can be overlaid or co-registered with the anatomical structures to improve diagnostic accuracy. The applications of nuclear medicine imaging techniques can include bone scanning which traditionally has had a strong role in the work-up/staging of cancers. Myocardial perfusion imaging is a sensitive and specific screening exam for reversible myocardial ischemia. Molecular imaging is the new and exciting frontier in this field.

The term “positron emission tomography (PET)” relates to a scanning method of nuclear medicine imaging. In PET scanning, a radioactive, biologically active substance, most often fluorine-18 fluorodeoxyglucose, is injected into a patient and the radiation emitted by the patient is detected to produce multi-planar images of the body. Metabolically more active tissues, such as cancer, concentrate the active substance more than normal tissues. PET images can be combined (or “fused”) with an anatomic imaging study (currently generally CT images), to more accurately localize PET findings and thereby improve diagnostic accuracy.

The term “single photon emission computed tomography” (SPECT) relates to a scanning method of nuclear medicine imaging using gamma rays. It is very similar to conventional nuclear medicine planar imaging using a gamma camera. However, it is able to provide true 3D information. This information is typically presented as cross-sectional slices through the patient, but can be freely reformatted or manipulated as required. The basic technique requires injection of a gamma-emitting radioisotope (radionuclide) into the bloodstream of the patient. Occasionally, the radioisotope is a simple soluble dissolved ion, such as a radioisotope of gallium (III), which happens to also have chemical properties which allow it to be concentrated in ways of medical interest for disease detection. However, most of the time in SPECT, a marker radioisotope, which is of interest only for its radioactive properties, has been attached to a special radioligand, which is of interest for its chemical binding properties to certain types of tissues. This enables the combination of ligand and radioisotope (the radiopharmaceutical) to be carried and bound to a place of interest in the body, which then (due to the gamma-emission of the isotope) allows the ligand concentration to be seen by a gamma-camera.

The term “optical techniques” relates to optical imaging via light reflector, fluorescent dyes, or sources of luminescence, optical imaging probes, near-infrared probes, bioluminescence, fluorescent proteins, green fluorescent protein, red fluorescent protein, yellow fluorescent proteins, luciferases, cytochromes, photoacoustic detection methods.

The present invention further relates to an embolization material comprising a magnetic particle composition having improved imaging properties and being detectable via magnetic particle imaging as described further herein below. In accordance with the present invention, the said magnetic particle composition comprises e.g. paramagnetic materials, and/or SPIOs or USPIOs.

Moreover, the present invention also contemplates the use of embolization material comprising metallic components, e.g. iron oxide for detection via magnetic particle imaging in combination with X-ray imaging, e.g. in a combined magnetic particle detection and X-ray detection interventional environment. Thus, acquisition of two- or three-dimensional distribution information of the embolization material is enabled via X-ray as well as via magnetic particle imaging.

Magnetic particle imaging as used in accordance with the present invention relates to an image technique wherein the embolization material is localized via changing of magnetic field, or radio waves, Gleich, Weizenecker “Tomographic imaging using the nonlinear response of magnetic particles”, Nature, 2005, 435, 1214-1217; Gleich et al., “Experimental results on fast 2D-encoded magnetic particle imaging”, Phys Med Biol, 2008, 53, N81-N84; Weizenecker et al., “Three-dimensional real-time in vivo magnetic particle imaging”, Phys Med Biol, 2009, 54, L1-L10). Basically, the spatial distribution of magnetic particles is determined in an examination area of an object of examination comprising the following steps: a) generation of a magnetic field with a spatial distribution of the magnetic field strength such that the examination area consists of a first sub-area with lower magnetic field strength and a second sub-area with a higher magnetic field strength, b) change of the particularly relative spatial position of the two sub-areas in the area of examination or change of the magnetic field strength in the first sub-area so that the magnetization of the particles changes locally, c) acquisition of signals that depend on the magnetization in the area of examination influenced by this change by radiofrequency or magnetization, incl. oscillating magnetization, and d) Evaluation of signals to obtain information about the change in spatial distribution and/or the movement of the magnetic particles in the area of examination, where the magnetic particles are introduced into and/or are present in the area of examination in a suspension, aerosol, in the form of a powder, especially diluted, with a casing or, more particularly a thin coating, present in at least one capsule, or coupled to cells, particularly white or red blood corpuscles, immune cells, tumor cells or stem cells, or to ingredients, medication, antibodies, transplants or living organisms, or in the preliminary stage form, especially liquid.

### FIGURES

FIG. 1: FTIR spectrum of the P(MAETIB-GMA) microparticles. The P(MAOETIB-GMA) microparticles were prepared by suspension polymerization of 495 mg MAOETIB and 5 mg GMA according to the experimental section.

FIG. 2: Light microscope image of the P(MAETIB-GMA) microparticles.

FIG. 3: SEM images of the P(MAOETIB-GMA) core microparticles (A) and the γ-Fe₂O₃/P(MAOETIB-GMA) core-shell microparticles (C). Images (B) and (D) illustrate higher magnification of the highlighted areas shown in images (A) and (C), respectively.

FIG. 4: Size histogram of the magnetic γ-Fe₂O₃ nanoparticles coating on the surface of the P(MAOETIB-GMA) core microparticles.

FIG. 5: Room temperature magnetization loop of the magnetic γ-Fe₂O₃/P(MAOETIB-GMA) core-shell microparticles.

FIG. 6: MR (A) and CT (B) imaging of a dual modality γ-Fe₂O₃/P(MAOETIB-GMA) core-shell microparticle in a rat's kidney.

FIG. 7: Histological images of a slice containing two acutely clotted vessels (A) and four slices containing vessels of the embolized rat's kidney blocked by the γ-Fe₂O₃/P(MAOETIB-GMA) microparticles (B).

FIG. 8: Scanning electron microscopy of multimodal embolization particles, inserts on the left mark magnified areas on the right. The isolated P(MAOETIB-GMA) particle core (A) shows a smooth surface (B) in contrast to coated particles (C) which show a rough surface (D) caused by 150 nm sized UPSIO particles.

FIG. 9: Multimodal embolization particles in ex-vivo imaging conditions in agar gel scanned in CT (a), MRI (b), angiography (c) and as a photograph (d). The particles are visible in all three imaging modalities in a good contrast.

FIG. 10: Renal substraction angiogram a) before and b) after embolization. Embolization caused a complete perfusion stop of kidney parenchyma. Embolization particles are evident close to a segmental renal artery in the central part of the organ (arrow).

FIG. 11: CT (a+b), MR T2* weighted images (c+d) and angiography (e+f) before (a,c,e) and after (b,d,f) embolization. Hyperdense punctual areas in CT (b), hypointense confluent areas in MR (d) and punctual hyperdensities appear after embolization (as indicated by arrows).

FIG. 12: MRI of kidney before (a, c) and after embolization (b,d) in T2 (a,b) weighted and EPI MRI (c,d) sequences (arrows).

FIG. 13: Coronar reformation through kidney after embolization. Position of signal changes after embolization correspond well on CT (A), MRI (B) and X-ray angiography (intraparenchymal, hilifugal columns, arrows) (C).

FIG. 14: Photography of one embolized kidney (A) showing sharp borders between ischemic, darker areas in the upper and lower pole. Histological images of embolized kidney parenchyma reveal particles (long arrows) residing in interlobular (B) and arcuate (C) arteries with consecutive thrombus.

FIG. 15: Scheme of a catheter through femoral artery via aorta up until kidney artery.

FIG. 16: Scheme of influx of particles (left: before injection, middle: while injection, right: after injection) via X-ray (upper row) and via nuclear spin imaging technique (lower row).

FIG. 17: Scheme of via particle (striated part) displaced vessel with consecutive thrombus development.

The following examples are only intended to illustrate the present invention. They shall not limit the scope of the invention in any way.

### EXAMPLE 1

The examined embolization material results in occlusion, e.g. occlusion of specific vessels, occlusion of bile ducts, or fistulae, and the treatment of aneurysms. This is achieved by adjusting size, stability, structure and/or (inflammation-) stimuli triggering features of the embolization material. Size can be variably adjusted in order to directly target different vessel regions (e.g. big or small tumor vessels).

The embolization material according to the present invention comprises of a combination of substances (and/or exhibits a combination of characteristic features) enabling detection of said material with more than one imaging technique. The characteristic features enable detection with appropriate sensitivity enabling good contrast under realistic conditions via at least two or more imaging techniques.

The embolization material comprises in addition to imaging inert materials at least substances of the following group
at least one radio-opaque element (such as e.g. calcium, iron, iodine, xenon, barium, ytterbium, gold, or bismuth) detectable via X-ray imaging
at least one paramagnetic element such as gadolinium detectable via MRI, or elements such as iron or compounds such as e.g. iron oxide (also in form of ultra small particles, e.g. ultra small paramagnetic iron oxid (USPIO) being paramagnetic or susceptible for MRI detection

The embolization material generally has at least two of the following characteristic features:
detectable via sonography, e.g. a gas, vacuum, or gas bubble to be filled with something using different ways,
detectable via nuclear medical imaging techniques, e.g. alpha-, beta-, or gamma-ray source of radiation; e.g. decaying atoms.
detectable via optical techniques, e.g. via light reflector, fluorescent dyes, or sources of luminescence, optical imaging probes, near-infrared probes, bioluminescence, fluorescent proteins, green fluorescent protein, red fluorescent protein, yellow fluorescent proteins, luciferases, cytochromes, photoacoustic detection methods,
detectable via radio waves (magnetic particle imaging), e.g. oscillating circuits, inductors, capacitors, or antennas, or emitters.

Multimodality visible materials (like other embolization materials) can be combined with chemotherapeutic agents or other therapeutic visible substances

### EXAMPLE 2

### Synthesis and Characterization of Dual Modality (CT/MRI) Core-Shell Microparticles for Embolization Purposes (Hagit et al., “Synthesis and Characterization of Dual Modality (CT/MRI) Core-Shell Microparticles for Embolization Purposes”, Biomacromolecules, 2010, 11(6): 1600-7).

Core P(MAOETIB-GMA) microparticles of 40-200 μm were prepared by suspension copolymerization of the iodinated monomer 2-methacryloyloxyethyl (2,3,5-triiodobenzoate), MAOETIB, with a low concentration of the monomer glycidyl methacrylate, GMA, which formed hydrophilic surfaces on the particles. Magnetic γ-Fe₂O₃/P(MAOETIB-GMA) core-shell microparticles were prepared by coating the aforementioned core particles through nucleation of iron oxide nanoparticles on the surfaces of the P(MAOETIB-GMA) particles. This was followed by stepwise growth of thin iron oxide layers. The radio-opacity and magnetism of these particles were demonstrated in vitro by CT and MRI. In vivo embolization capabilities of these first multimodal visible embolization particles were demonstrated in a rat's kidney tumor embolization model.

### 2.1. Materials

The following analytical-grade chemicals were purchased from Aldrich-Sigma and used without further purification: 2,3,5-triiodobenzoic acid (98%), HEMA (99%), 1,3-dicyclohexylcarbodiimide (DCC, 99%), 4-pyrrolidinopyridine (98%), diethyl ether anhydrous (99.7%), MgSO₄ (99%), ethyl acetate (99.5%), GMA (97%), benzoyl peroxide (BP, 98%), polyvinylpyrrolidone (PVP, m.w. 360,000), ferrous chloride tetrahydrate (Fluka), sodium hydroxide (standard solution 1M), sodium nitrite (99.9%), toluene (HPLC grade), dextrose (99.5%), agarose low EEO, formaldehyde (99.9%), isopropanol (99.5%), xylene (99%), paraffin, hematoxylin and eosin (90%). Gd-DOTA was purchased from Dotarem®, Guerbet, France. Water was purified by passing deionized water through an Elgastat Spectrum reverse osmosis system (Elga Ltd., High Wycombe, UK).

### 2.2. Methods

### 2.2.1. Synthesis of MAOETIB

The iodinated monomer MAOETIB was synthesized according to following scheme.

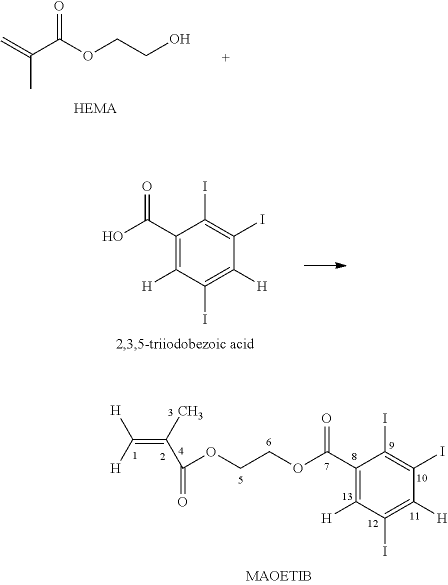

Briefly, 2,3,5-triiodobenzoic acid (50 g, 0.10 mol), HEMA (15 g, 0.11 mol), DCC (23 g, 0.11 mol) and 4-pyrrilidinopyridine (1.5 g, 0.010 mol) were dispersed in ether (500 ml), and then stirred at room temperature for 18 h. The formed solid was filtered off and the residue washed with fresh ether. The ether solution was then washed with HCl (2 N) and saturated NaHCO₃. The organic phase was dried over MgSO₄, filtered, and evaporated to produce an orange solid. Pure white crystals of MAOETIB (m.p. 95° C.) were obtained by the two-fold recrystallization of the orange solid from ethyl acetate (yield 84%).

The following spectra were obtained:

¹H NMR (CDCl₃) δ 1.97 (s, 3H, CH₃), 4.57 and 4.48 (m, 4H, OCH₂CH₂O), 5.61 (s, 1H, olefinic), 6.16 (s, 1H, olefinic), 7.33 (d, 1H, J=1.68 Hz, Ar—H), 8.30 (d, 1H, J=1.68 Hz, Ar—H).

¹³C NMR (CDCl₃) δ 18.33 (C-3), 61.92 (C-5), 63.93 (C-6), 93.64 (C-12), 106.56 (C-9), 113.39 (C-10), 126.41 (C-1), 135.72 (C-2), 137.13 (C-13), 148.86 (C-11), 165.60 (C-4), 166.97 (C-7). MS (ES+): m/z 635 (MNa⁺, 100).

The molecular weight of the monomer, MAOETIB, was confirmed by mass spectrometry. Elemental analysis—Calculated: C, 25.52; H, 1.81; O, 10.46; I, 62.21. Experimental: C, 25.65; H, 1.82; O, 10.49; I, 62.04.

### 2.2.2. Synthesis of the PMAOETIB Microparticles

PMAOETIB microparticles were prepared by suspension polymerization of MAOETIB according to the following procedure: 10 ml of toluene solution containing 0.5 g MAOETIB and 40 mg BP (8% w/wₘₒₙₒₘₑᵣ) were introduced into a flask containing 100 ml of 1% PVP aqueous solution. The mixture was then stirred at 80° C. for 15 h. The organic phase containing the toluene and excess monomer was then extracted from the aqueous phase. The formed PMAOETIB microparticles were then washed by extensive centrifugation cycles with water and then dried by lyophilization.

### 2.2.3. Synthesis of the P(MAOETIB-GMA) Core Microparticles

P(MAOETIB-GMA) copolymeric microparticles were prepared by suspension polymerization of MAOETIB and GMA according to the following procedure: 10 ml of toluene solution containing 495 mg MAOETIB, 5 mg GMA and 40 mg BP (8% w/wₘₒₙₒₘₑᵣ) were introduced into a flask containing 100 ml of 1% PVP aqueous solution. The mixture was then stirred at 80° C. for 15 h. The organic phase containing the toluene and excess monomer was then extracted from the aqueous phase. The formed P(MAOETIB-GMA) microparticles were then washed by extensive centrifugation cycles with water and then dried by lyophilization. The dried microparticles were then sieved in fractions of sizes ranging between 40-200 μm.

### 2.2.4. Synthesis of the γ-Fe₂O₃/P(MAOETIB-GMA) Core-Shell Microparticles

Magnetic γ-Fe₂O₃/P(MAOETIB-GMA) core-shell microparticles were prepared by coating the P(MAOETIB-GMA) microparticles with successive layers of the γ-Fe₂O₃ nanoparticles according to the following procedure: an aqueous suspension containing 300 mg of the P(MAOETIB-GMA) microparticles in 300 ml of distilled water was mechanically stirred at 60° C. Nitrogen was bubbled through the microparticles aqueous suspension during the coating process to exclude air. Volumes of 0.5 ml of reference aqueous solutions of FeCl₂.4H₂O (25 mM) and 0.5 ml of NaNO₂ (1.5 mM) were successively introduced into the reaction flask. Then, an aqueous solution of sodium hydroxide (50 mM) was added until a pH of about 9.5 was reached. The mixture was then stirred for 1 h. This procedure was repeated 10 times. During this coating process, the surface of the P(MAOETIB-GMA) microparticles became brown-black in color. The suspension was then cooled to room temperature under nitrogen atmosphere. The microparticles produced were washed extensively in water and then dried by lyophilization.

### 2.2.5. In Vitro CT and MRI Imaging

The dual modality γ-Fe₂O₃/P(MAOETIB-GMA) microparticles were inserted (via 21 G needles) in several sites of an agarose gel on a Petri dish. CT and MRI scans were then performed.

### 2.2.6. In Vivo CT and MR Imaging

A male Copenhagen rat (weighing approx. 500 g) was used to evaluate the embolization capability as well as in-vivo X-ray and MRI signal change of the γ-Fe₂O₃/P(MAOETIB-GMA) microparticles. The experiment was approved by the German governmental committee on animal care. Gas narcosis was started before the first manipulation of the animal and maintained until death of the animal.

A catheter was inserted into the right femoral artery, the catheter tip placed in proximity to the left renal artery. During injection, the aorta was ligated above and below the left renal artery, to prevent particles from diverting to other organs. A 5% dextrose aqueous dispersion containing 3 mg of the γ-Fe₂O₃/P(MAOETIB-GMA) microparticles was injected through the catheter. CT and MRI scans were performed. After scanning, the rat was killed and the left kidney was removed for histology.

### 2.3. Characterization

¹H and ¹³C NMR spectra were obtained with a Bruker DPX-300 spectrometer. Chloroform-d and tetrahydrofuran-d₈ (THF-d₈) chemical shifts are expressed in ppm downfield from tetramethylsilane used as an internal standard.

Mass spectra were obtained with a Finnigan 4021 spectrometer (electrospray and desorption chemical ionization).

Fourier Transform Infrared (FTIR) analysis was performed with a Bomem FTIR spectrophotometer, Model MB 100, Hartman & Braun. The analysis was performed with 13 mm KBr pellets that contained 2 mg of the examined particles and 198 mg of KBr. The pellets were scanned over 200 scans at a 4 cm⁻¹ resolution.

Optical microscope pictures were obtained with an Olympus microscope, model BX51. Surface morphology was characterized with a FEI scanning electron microscope (SEM) model Inspect S. For this purpose, a drop of dilute microsphere dispersion in water was spread on a glass surface, and then dried at room temperature. The dried sample was coated with gold in vacuum before viewing under SEM.

Dry size and size distribution were determined by measuring the diameter of more than 100 particles observed under SEM with image analysis software, AnalySIS Auto (Soft Imaging System GmbH, Germany)

Elemental analysis (C, O, Fe and I) was performed by the analytical services of the Microanalysis Lab, of the Institute of Chemistry, the Hebrew University of Jerusalem, Jerusalem. The reported values are an average of measurements performed on at least three samples of each of the tested particles, and have a maximum error of about 2%.

Surface elemental analysis was obtained by X-ray photoelectron spectroscopy (XPS), Model AXIS-HS, Kratos Analytical, England, using Al K_{α}, lines, at 10⁻⁹ Torr, with a takeoff angle of 90°. The reported elemental values of the XPS are an average of measurements performed at least four times for each of the tested particles, and have a maximum error of about 5%.

The surface area of the various particles was measured by the Brunauer-Emmet-Teller (BET) method with Gemini III model 2375, Micrometrics. The reported values of the surface area are an average of measurements performed at least four times for each of the tested particles.

Magnetic measurements were performed on a sample of dried particles that was introduced into a plastic capsule. Magnetization was measured as a function of the external field being swept up and down (−14,000 Oe<Happlied<14,000 Oe, in steps of 200 Oe).

Agar phantoms were prepared using 2% Agar supplemented with 0.2 ml/l Gd-DOTA. 21 G needles were used to insert the γ-Fe₂O₃/P(MAOETIB-GMA) microparticles in several sites as well as to create control injection canals in the absence of the particles.

MR imaging was performed with a whole-body MR scanner (3T Magnetom Tim Trio MRI, Siemens, Germany) and a small animal coil (Rapid Biomedical, Germany). The following scanning protocols have been used: A T2*-weighted GRE sequence for ex-vivo scans (repetition time 620 ms, echo time 20 ms, 320×260 matrix, 2 averages, flip angle 20°, pixel size 0.375×0.375 mm, slice thickness 2 mm) and a T1-weighted 3D GRE sequence for in-vivo scans (repetition time 18 ms, echo time 12 ms, 384×145 matrix, 2 averages, flip angle 10°, pixel size 0.2×0.2 mm, slice thickness 0.7 mm).

CT imaging was performed with clinical CT scanner (Dual Source Definition CT, Siemens, Germany) using the following parameters of a high-resolution spiral scan: 80 kV, 32 mA, H41 kernel, 32 detector rows with a total collimation of 19.2 mm, a spiral pitch of 0.9 and a recon slice thickness of 0.5 mm. For histological examination, 3 μm slices were obtained after fixation, dehydration and embedding of the tissue using formaldehyde, isopropanol, xylene and paraffin. Slices were then stained by hematoxylin & eosin.

### 2.3.1. Characterization of the P(MAOETIB-GMA) Microparticles

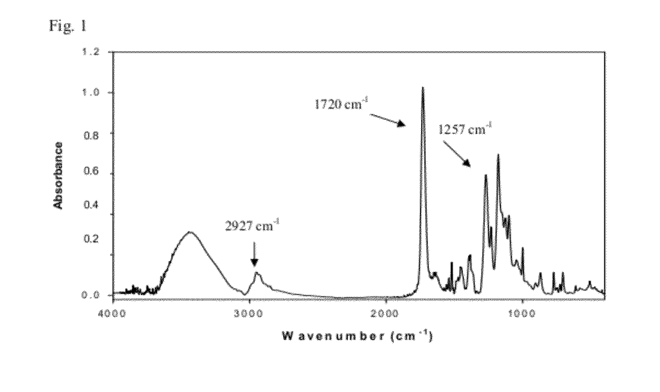

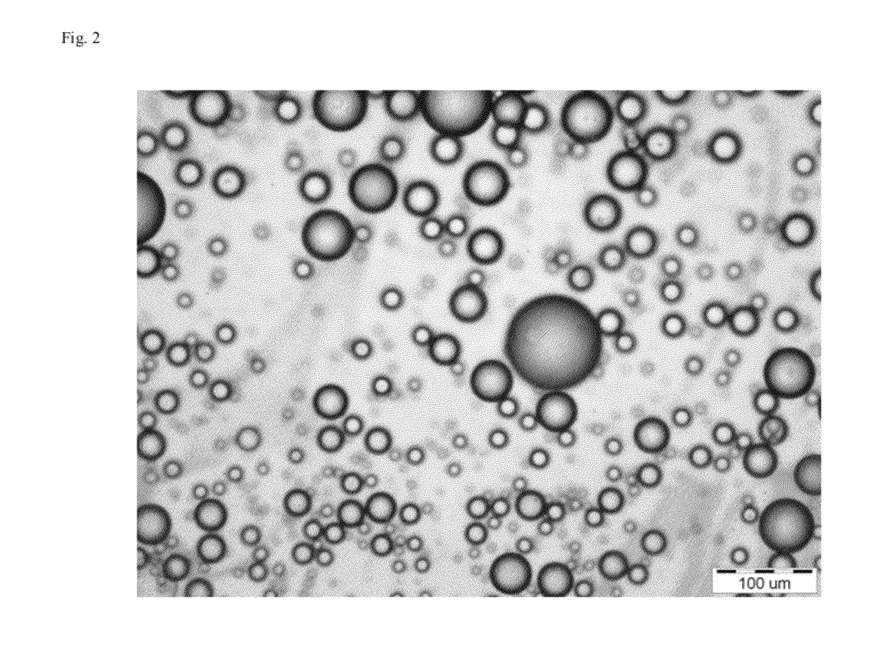

FIG. 1 presents the FTIR spectrum of the P(MAOETIB-GMA) microparticles. The FTIR spectrum displays absorption peaks at 1720 cm⁻¹ corresponding to the carbonyl group stretching bands, 1257 cm⁻¹ corresponding to the ester bond stretching bands, 2851, 2927 and 2976 cm⁻¹ corresponding to the aromatic CH stretching bands. The P(MAOETIB-GMA) microparticles were free of traces of the monomer, as was verified by ¹H-NMR (THF-d₈), by the lack of the two peaks of the vinylic protons at 5.61 and 6.16 ppm, and by FTIR, by the lack of the C═C double-bond stretching band at about 1623 cm⁻¹. The FTIR spectrum of the P(MAOETIB-GMA) copolymeric microparticles did not show peaks at 845 and 910 cm⁻¹ corresponding to the epoxide vibrational bands of the GMA monomeric units. We therefore assume that under the experimental conditions, each of the epoxide groups splits open to two hydroxyl groups. Indeed, the FTIR spectrum of the P(MAOETIB-GMA) microparticles indicated a clear typical absorption peak of the —OH stretching band at about 3500 cm⁻¹. FIG. 2 presents by a light microscope image the relatively broad size distribution of the formed P(MAETIB-GMA) microparticles obtained via the suspension copolymerization of the monomers MAOETIB and GMA. Therefore, we use sieves of various sizes in order to narrow the size distribution of these microparticles, to between 40-200 μm.

### 2.3.2. Bulk and Surface Analysis and Composition of the P(MAOETIB-GMA) and the PMAOETIB Microparticles

Table 1 presents the elemental analysis data of the P(MAOETIB-GMA) and the PMAOETIB microparticles, and the fraction composition (weight % of the polymerized MAOETIB units and the polymerized GMA units) of these microparticles. The weight % of the polymerized MAOETIB units of the copolymeric microparticles was calculated from the following equation:

% polymerized MAOETIB=[(% I_{P(MAOETIB-GMA)}×100)]/% I_{PMAOETIB}

The weight % of I_{P(MAOETIB-GMA)} and I_{PMAOETIB} were obtained from the iodine elemental analysis of the relevant microparticles. The weight % of polymerized GMA units was calculated by subtracting the weight % of the polymerized MAOETIB units from 100. The elemental analysis provides information on the bulk composition of the microparticles. Table 1 indicates that the iodine content of the P(MAOETIB-GMA) copolymeric microparticles is just slightly less than that of the PMAOETIB homopolymeric microparticles, 56.9 and 58.2%, respectively. Table 1 also indicates that the copolymeric microparticles are composed of 97.7% polymerized MAOETIB and 2.3% polymerized GMA. This fractional composition has a very good correlation with the initial concentration of MAOETIB and GMA used for this copolymerization, 99 and 1%, respectively. It should be noted that the composition of the copolymeric microparticles shown in Table 1 is not precise since the calculations do not take into account the initiator and stabilizer fractions of the polymeric chains belonging to the copolymeric microparticles. Similarly, the PMAOETIB microparticles contain 58.2% iodine (Table 1) while the monomer contains 62%. This slightly lower iodine content of the PMAOETIB microparticles and of the pure PMAOETIB polymer is probably due to the initiator fraction of the polymeric chains belonging to the microparticles.

Table 2 presents the elemental surface analysis of the P(MAOETIB-GMA) and the PMAOETIB microparticles, as measured by XPS, and the calculated surface fraction composition of these microparticles. The surface weight % of the polymerized MAOETIB units of the copolymeric microparticles was calculated according to the previous equation, substituting the elemental analysis data for the XPS data. The surface weight % of the polymerized GMA units of the copolymeric microparticles was calculated by reducing the weight % of the surface polymerized MAOETIB units from 100. XPS measurements provide information on the most outer surface composition of the iodinated microparticles, while the elemental analysis provides similar information on the bulk composition of these microparticles. Table 2 illustrates that the surface iodine content of the copolymeric P(MAOETIB-GMA) microparticles is significantly lower than that of the PMAOETIB, 0.6 and 47.3%, respectively. This iodine content illustrates that the surface of the P(MAOETIB-GMA) microparticles is composed of 1.3% of polymerized MAOETIB units and 98.7% of the reduced polymerized GMA units (which also includes the initiator and stabilizer fractions). This significant decrease in the % of the surface polymerized MAOETIB units fraction compared to the bulk fraction (1.3 and 97.7%, respectively) and the increase in the surface polymerized GMA fraction (98.7 and 2.3%, respectively) indicates that most of the reduced polymerized GMA units part of the P(MAOETIB-GMA) chains reconstruct on the surface of the copolymeric microparticles dispersed in the aqueous continuous phase. Despite neglecting the initiator and the stabilizer fractions of the non-iodinated material, this conclusion is based mainly on the iodine content and therefore is accurate.

TABLE 1
Elemental analysis and bulk composition of the P(MAOETIB-
GMA) and the PMAOETIB microparticles.^{a}

| | Weight % | | | | |
|---|---|---|---|---|---|
| | | | | Polymerized | Polymerized |
| Microparticles | C | O | I | MAOETIB units | GMA units |
| P(MAOETIB- | 29.7 | 10.2 | 56.9 | 97.7 | 2.3 |
| GMA) | | | | | |
| PMAOETIB | 28.3 | 11.5 | 58.2 | 100 | 0 |

^{a}The P(MAOETIB-GMA) microparticles were prepared by suspension polymerization of 495 mg MAOETIB and 5 mg GMA according to the experimental section. The PMAOETIB microparticles were prepared by suspension polymerization of 500 mg MAOETIB according to the experimental section.

TABLE 2
Surface elemental analysis and composition of the
P(MAOETIB-GMA) and the PMAOETIB microparticles.^{a}

| | Surface weight % | | | | |
|---|---|---|---|---|---|
| | | | | Polymerized | Polymerized |
| Microparticles | C | O | I | MAOETIB units | GMA units |
| P(MAOETIB- | 65.8 | 33.6 | 0.6 | 1.3 | 98.7 |
| GMA) | | | | | |
| PMAOETIB | 40.9 | 11.8 | 47.3 | 100 | 0 |

^{a}The P(MAOETIB-GMA) microparticles were prepared by suspension polymerization of 495 mg MAOETIB and 5 mg GMA according to the experimental section. The PMAOETIB microparticles were prepared by suspension polymerization of 500 mg MAOETIB according to the experimental section.

2.3.3. Coating of the Copolymeric P(MAOETIB-GMA) Microparticles with the Magnetic γ-Fe₂O₃ Nanoparticles

The use of the GMA was essential in order to create a hydrophilic surface on the iodinated copolymeric particles. This hydrophilic surface enables the coating of the magnetic iron oxide nanoparticles onto the core P(MAOETIB-GMA) particles. The attachment of the magnetic nanoparticles to the surface of the iodinated copolymeric particles is strong and they do not leach from the surface of the particles to the aqueous continuous phase. This was illustrated by separating the core-shell microparticles from the continuous aqueous phase by centrifugation, followed by confirming the absence of iron oxide nanoparticles in the supernatant.

The weight % of the γ-Fe₂O₃ coating on the copolymeric particles was calculated from the % Fe obtained by elemental analysis. Since the P(MAOETIB-GMA) core microparticles do not contain Fe, the % Fe may be used for calculating the weight % of the γ-Fe₂O₃ coating on the γ-Fe₂O₃/P(MAOETIB-GMA) core-shell microparticles, according to the following equation: % γ-Fe₂O₃=(% Fe×100)/69.8, where % Fe is obtained from the elemental analysis, and 69.8 is the % Fe in pure γ-Fe₂O₃. Fe analysis of the γ-Fe₂O₃/P(MAOETIB-GMA) microparticles illustrated that under the experimental conditions the % Fe is 2.4, thereby the calculated % γ-Fe₂O₃ of these core-shell microparticles is 3.4%.

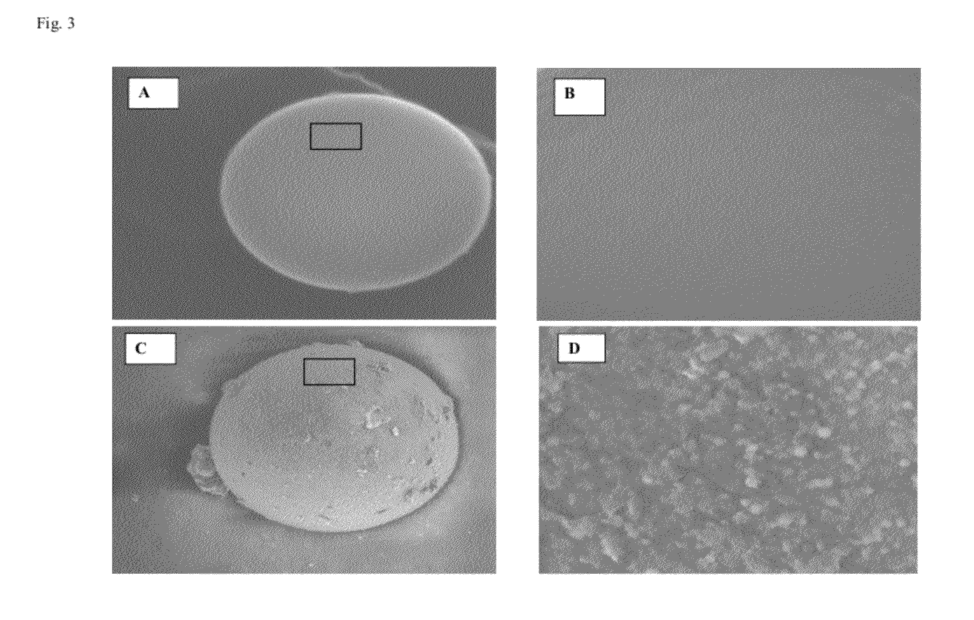

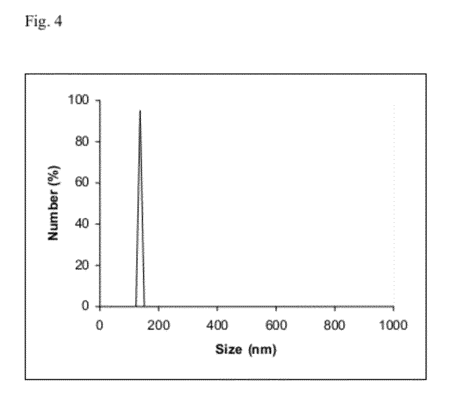

FIG. 3 presents SEM images of the P(MAOETIB-GMA) core microparticles (A), and the γ-Fe₂O₃/P(MAOETIB-GMA) core-shell microparticles (C). Images (B) and (D) illustrate higher magnification of the highlighted areas shown in images (A) and (C), respectively. FIG. 3(B) indicates that the surface of the P(MAOETIB-GMA) microparticles is smooth while the surface of the γ-Fe₂O₃/P(MAOETIB-GMA) microparticles is bumpy and uniformly coated with the magnetic nanoparticles as observed in FIG. 3(D). FIG. 4 illustrates that the size and size distribution of the γ-Fe₂O₃ nanoparticles coated on the surface of the P(MAOETIB-GMA) microparticles is 137±15 nm. BET surface area of the P(MAOETIB-GMA) microparticles and the magnetic γ-Fe₂O₃/P(MAOETIB-GMA) core-shell microparticles showed an increased measured surface area of the γ-Fe₂O₃/P(MAOETIB-GMA) microparticles (1.71±0.06 m²/g) compared to that of the P(MAOETIB-GMA) microparticles (0.82±0.05 m²/g). This difference can probably be attributed to the bumpy roughened surface of the magnetic γ-Fe₂O₃/P(MAOETIB-GMA) core-shell microparticles (FIG. 3C) as compared to the smooth surface of the P(MAOETIB-GMA) microparticles (FIG. 3A).

The magnetic γ-Fe₂O₃/P(MAOETIB-GMA) core-shell microparticles were prepared by coating the P(MAOETIB-GMA) core microparticles with the magnetic iron oxide nanoparticles according to the experimental section.

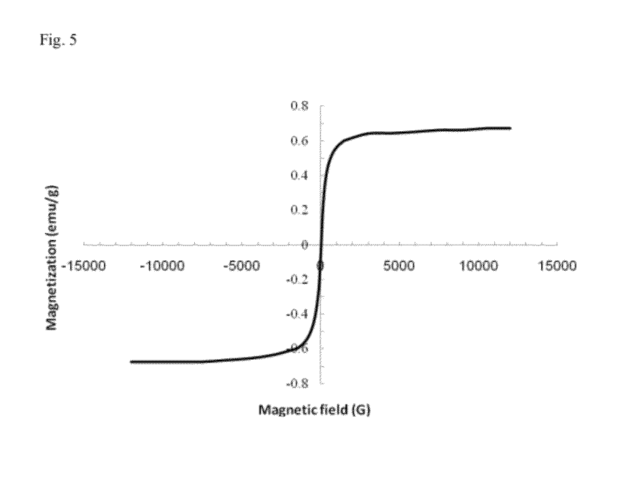

FIG. 5 presents the magnetization curve of the γ-Fe₂O₃/P(MAOETIB-GMA) core-shell microparticles after reducing the control curve of the P(MAOETIB-GMA) microparticles. At room temperature, the particles show a relatively low magnetic saturation of 0.67 emu/g. This low magnetic moment is due to the relatively low content of the magnetic nanoparticles belonging to the P(MAOETIB-GMA) microparticles. FIG. 5 also indicates that at room temperature the magnetization loop does not show any hysteresis. Although the saturation magnetization (MS) value is low, these particles respond rapidly even to a low magnetic field such as 100 Gauss. The observed merging temperature of the ZFC/FC curves of the γ-Fe₂O₃/P(MAOETIB-GMA) microparticles was 305° K, indicating that the magnetic nanoparticles on the surface of the P(MAOETIB-GMA) microparticles exhibit ferromagnetic behavior.

The magnetic γ-Fe₂O₃/P(MAOETIB-GMA) core-shell microparticles were prepared by coating the P(MAOETIB-GMA) core microparticles with the magnetic iron oxide nanoparticles according to the experimental section.

### 2.3.4. In Vitro X-Ray and MRI Visibility of the γ-Fe₂O₃/P(MAOETIB-GMA) Microparticles

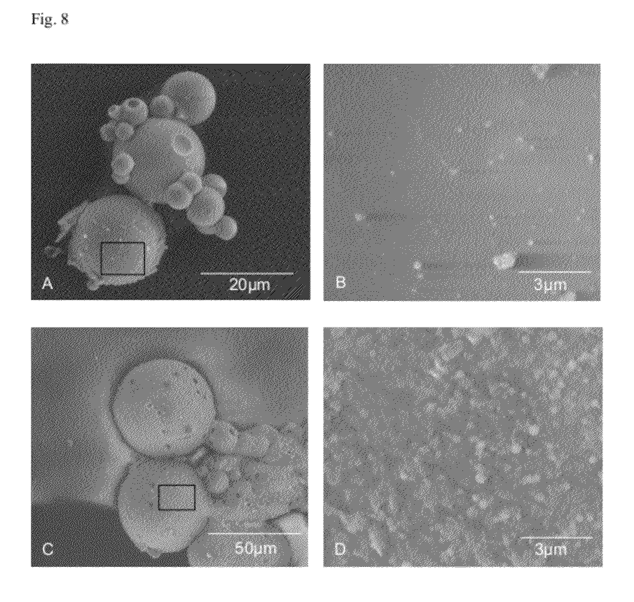

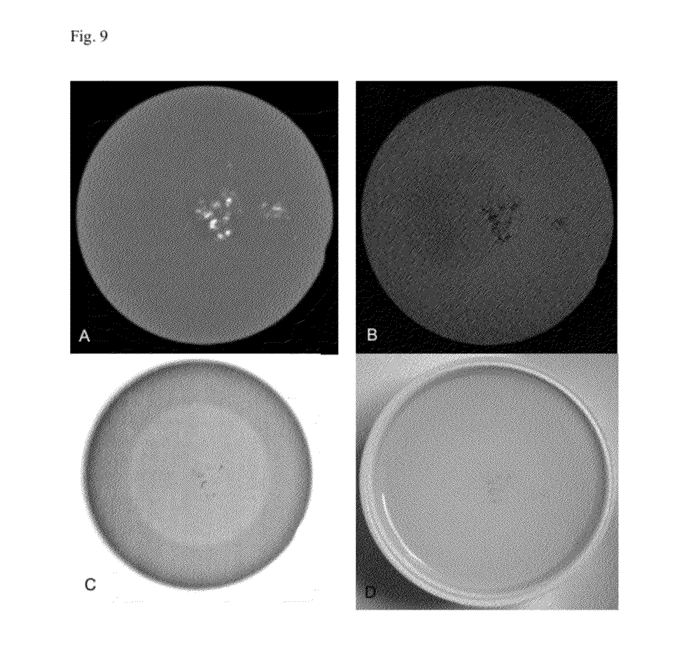

An illustration of the in vitro CT (A) and MR (B) imaging of the dual modality γ-Fe₂O₃/P(MAOETIB-GMA) core-shell microparticles is presented in FIG. 9 A, B. For this purpose the microparticles were inserted into an agarose gel placed in a petri dish. It is clear from the location of the particle, in the images of FIG. 9 A, B, that this dual modality particle is visible in both imaging techniques. The iodine content enables the enhancement in CT and the iron oxide content enables the signal void in MRI. Similar images were also obtained for several microparticles within the agarose gel.

The magnetic γ-Fe₂O₃/P(MAOETIB-GMA) core-shell microparticles were prepared by coating the P(MAOETIB-GMA) core microparticles with the magnetic iron oxide nanoparticles. The γ-Fe₂O₃/P(MAOETIB-GMA) microparticles were inserted to an agarose gel placed on a petri dish. CT and MRI scans were then performed according to the experimental section.

### 2.3.5. In Vivo X-Ray and MRI Visibility of Rat's Kidney Embolized by the γ-Fe₂O₃/P(MAOETIB-GMA) Microparticles

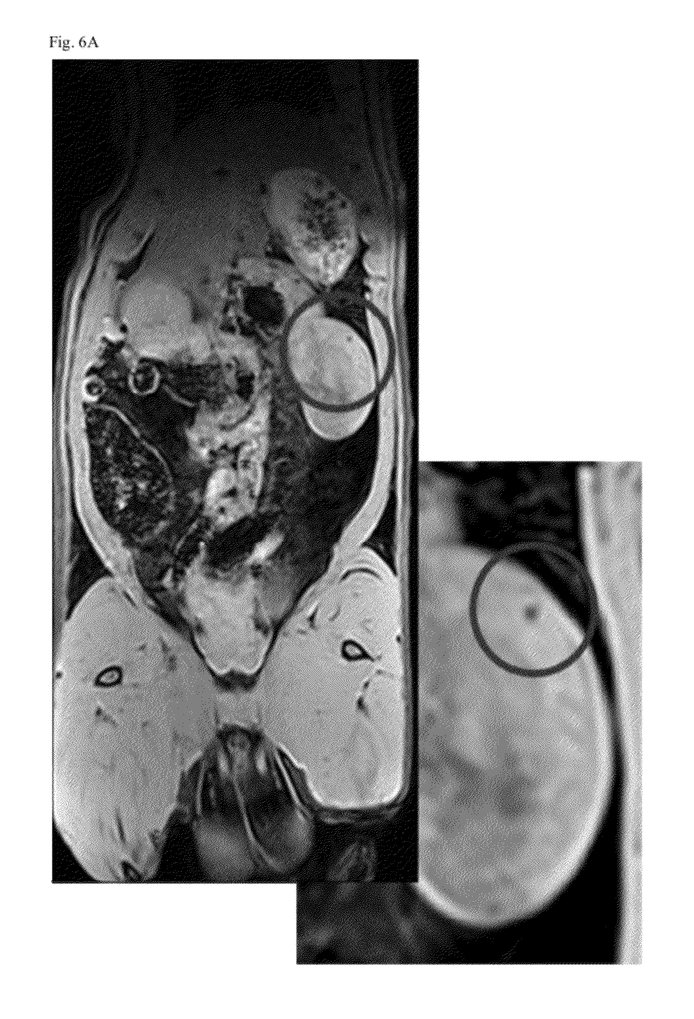

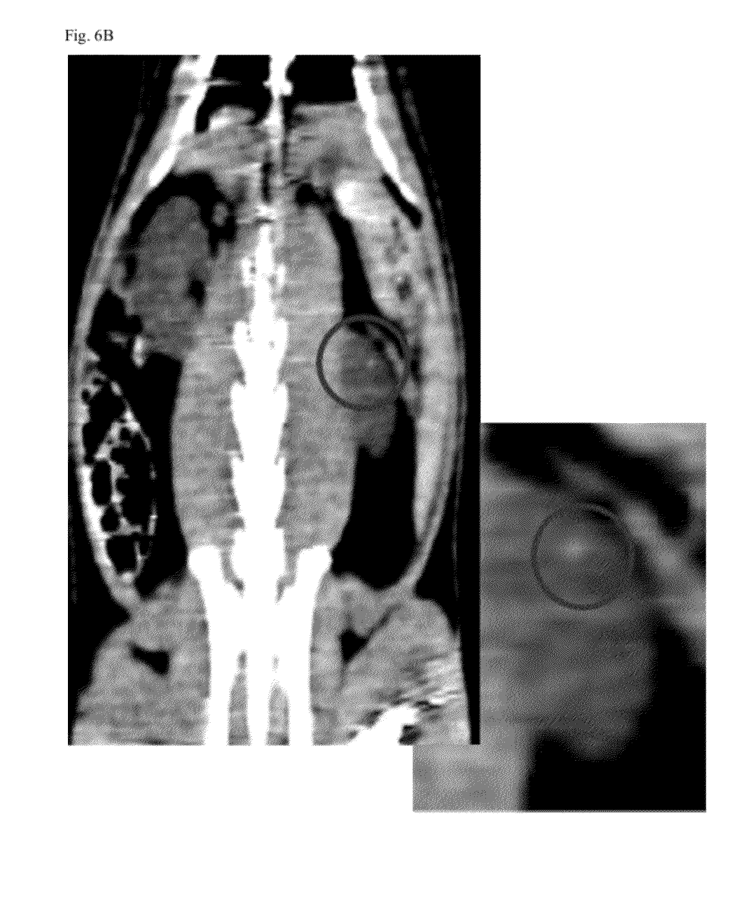

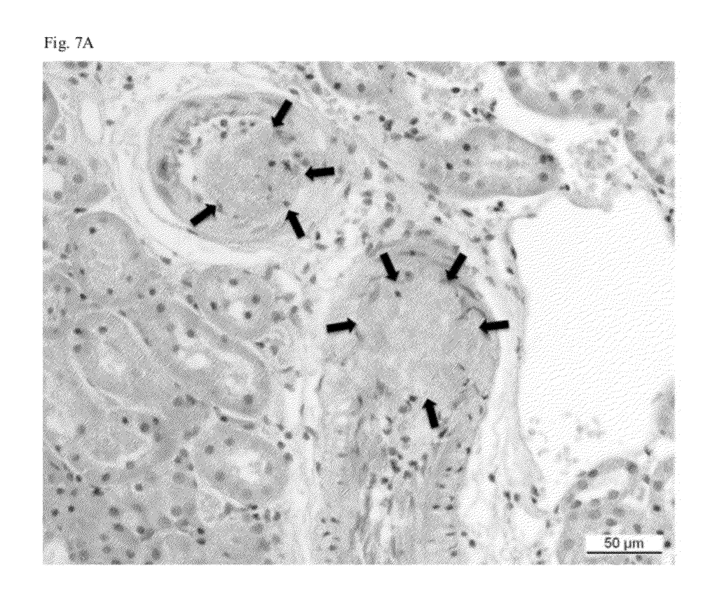

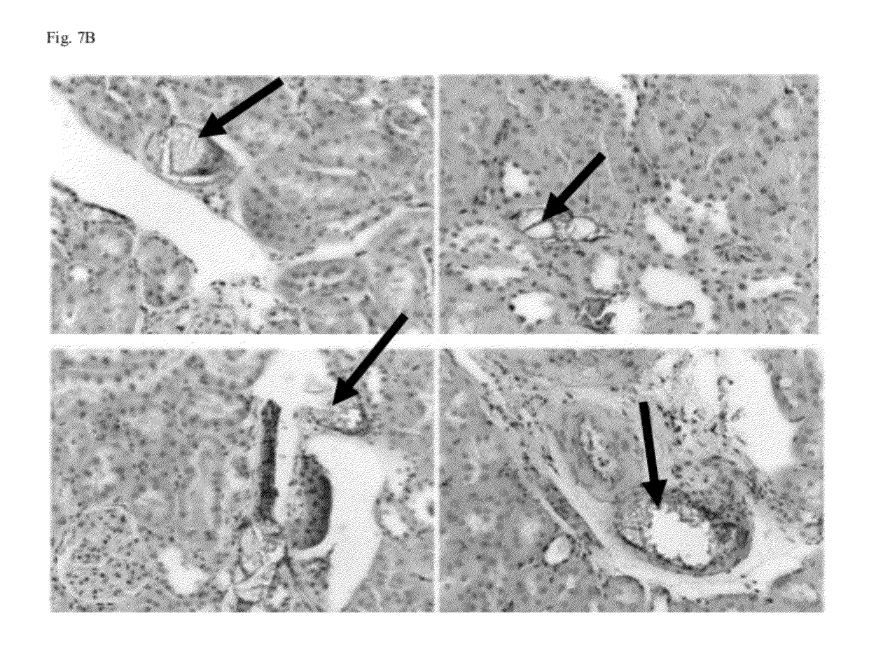

FIG. 6 presents in vivo MRI (A) and CT (B) imaging of the dual modality γ-Fe₂O₃/P(MAOETIB-GMA) core-shell microparticle after injection of a 5% dextrose aqueous dispersion containing 3 mg of the γ-Fe₂O₃/P(MAOETIB-GMA) microparticles to the catheter tip. A signal change in both MRI and CT of a γ-Fe₂O₃/P(MAOETIB-GMA) microparticle blocking a vessel in the rat's kidney in corresponding areas is observed in the circled areas. The size of the signal changes in the kidney was measured and found to be 70 μm which correlates to the size of a single particle. This figure presents a thin slice, in other slices, similar signals were observed. After scanning the kidney in vivo by both MRI and CT, the rat was killed and the left kidney was removed for histology. FIG. 7 presents histologic images of a kidney slice containing two acutely clotted vessels surrounded by black arrows (A) and four kidney slices containing the γ-Fe₂O₃/P(MAOETIB-GMA) microparticle (B), the black arrows point at the blocking microparticles. FIG. 7(A) shows a thrombus (fibrin/thrombocytes) in the kidney vessels caused by particles that were blocking the blood pool and are not found in that particular histological slice. In image 8(B), particles are observed while thrombus is not.

The magnetic γ-Fe₂O₃/P(MAOETIB-GMA) core-shell microparticles were prepared by coating the P(MAOETIB-GMA) core microparticles with the magnetic iron oxide nanoparticles according to the experimental section.

### 2.4. Results

The radiopaque magnetic γ-Fe₂O₃/P(MAOETIB-GMA) core-shell microparticles were synthesized by coating the P(MAOETIB-GMA) microparticles prepared by suspension polymerization with the γ-Fe₂O₃ nanoparticles. The magnetic shell coating on the surface of the radiopaque P(MAOETIB-GMA) core enables the microparticles to cause changes in both the MRI and CT signals. The embolization ability of the γ-Fe₂O₃/P(MAOETIB-GMA) core-shell microparticles has been proved by initial animal experiments. For the first time multimodal-visible particles designed for embolization therapy have been synthesized and successfully tested in-vivo.

### EXAMPLE 3

### First Multimodal Embolization Particles Visible on X-Ray/CT and MRI

### Materials and Methods

### Multimodal Embolization Particles

Multimodal embolization particles (FIG. 2) consist of an X-ray visible, iodine containing core and a MRI visible, ultra small paramagnetic iron oxide (USPIO)-based coating. The core was synthesized by suspension homopolymerization of 2-methacryloyloxyethyl (2,3,5-triiodobenzoate) (MAOETIB) together with a low concentration of glycidyl methacrylate (GMA). This resulted in a long polymer P(MAOETIB-GMA) with iodinated, aromatic side chains. This core was coated with Fe₃O₄ by nucleation and controlled growth mechanism of magnetic iron oxide nanoparticles on its surface, resulting in magnetic Fe₃O₄/P(MAOETIB-GMA) particles (FIG. 8).

For embolization, particles with diameters ranging from 40 to 200 μm were selected by multiple sieving steps. Adhesion effects due to the electric charge of the particles made it necessary to add 25 g/l rabbit albumin (Sigma Aldrich, Germany) to distilled water to disperse the particles and to prevent the particles from sticking to syringe and catheter walls and allow them to float freely in suspension.

### In-Vitro Imaging Characterization

A Petri dish was filled with a 2% agarose solution (Sigma Aldrich, Germany) to a height of 1.5 cm. After 30 min of cooling, particles were placed on the surface and then covered with a 1 cm layer of agar. The agar phantom was imaged within angiography (Table 3, No. 1), CT (Table 3, No. 2) and MRI (Table 3, No. 4).

### Embolization Animal Model

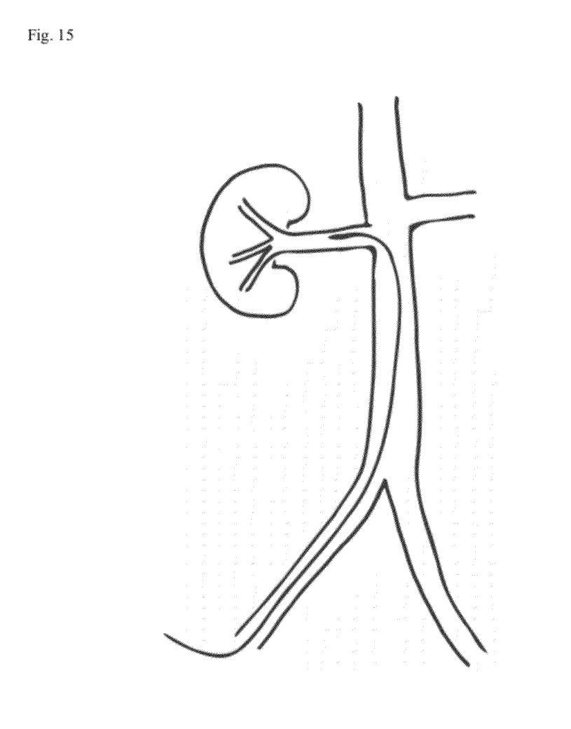

A standard, well-established tumor embolization animal model has been used to test the particles in-vivo (FIG. 15). Here, a rabbit kidney—representing a tumor—was embolized. All animal experiments were approved by the responsible local authorities. Six New Zealand White rabbits with an average weight of 3.4 kg (±0.8 kg) were used. Animal anesthesia was initiated and maintained using a combination of Diazepam (2.5 g/kg s.c.), Xylazinhydrochloride (Rompun®, 10 mg/kg i.m.) and Ketaminhydrochloride (Ketanest® 70 mg/kg i.m.). After additional local anesthesia using Mepicavaine, the right femoral artery was surgically exposed and a 16 G standard venous cannula was inserted. A hemostasis valve (Terumo hemostasis valve II, Terumo, Japan) was connected to the cannula. Heparin (1000 IU) was applied to prevent blood clot forming.

Using a 0.018″ microcatheter (MicroFerret-18, Cook Medical, USA) one kidney artery was catheterized, a bend micro guide wire (0.016″, Radiofocus Guidewire GT, Terumo, Japan) was used if necessary. In three cases the right kidney artery was probed, the downward oriented upbranching of the right kidney artery from the aorta facilitated catheterization of this side. In three cases the left kidney was selected, because it was assumed that the left kidney might be less prone to movement artifacts during MR imaging. Correct catheter positioning and normal kidney perfusion was confirmed by injecting 0.5 ml of iodinated contrast media during angiographic series acquisition (Table 3, No. 1).

### Study Design and Imaging

In all animals an angiographic times series (Table 3, No. 1), CT (Table 3, No. 2, 3) and MRI (Table 3, No. 5, 6, 7) was performed before and after embolization of the kidney. To demonstrate real-time visibility of the embolization process, additionally continuous imaging during embolization itself was performed in each modality in two animals (Table 4). In angiography a time series (Table 3, No. 1), in CT a continuously updated slap through the midsection of the kidneys were acquired during application of the particles (Table 3, No. 3). In MRI a fast, repetitive, coronal EPI (echo planar imaging) sequence (Table 3, No. 8) was used.

### Histology

After imaging, the animals were sacrificed and organs were taken and prepared for histology. Both kidneys were cut into eight 0.5 cm horizontal slices each. In addition, control slices of lung, liver and brain were taken. The samples were fixed in 4% formalin, dehydrated using Ethanol and Xylene and embedded in Paraffin. A microtome (Leica RM 2165, Leica, Germany) was used to obtain 3 μm slices. After Haematoxylin and Eosin staining, the slides were examined using bright field microscopy (DMREHC Microscope, Leica, Germany).

### Data Analysis

CT signal to noise ratio (SNR) was calculated using a two region of interest (ROI) approach. Two circular ROIs were placed in one slice, one ROI containing only particles, the other only plain agar. SNR was calculated using following formula:

$$S N R = \frac{\begin{matrix}( mean value of signal in particle ROI ) - \\ ( mean value of signal in agar ROI )\end{matrix}}{( standard deviation of signal in agar ROI )}$$

SNR was calculated in 5 different sites for single particles as well as for particle clusters.

For SNR measurements in MRI, summation and difference images were calculated out of two identical consecutively acquired T2 weighted scans (Table 3, No. 4). Corresponding ROIs containing particle signal in both summation and difference images were then taken to calculate the SNR of particles in 5 different representative sites using following formula:

$$S N R = \frac{1}{\sqrt{2}} \times \frac{( mean value of signal in sum image ROI )}{( \begin{matrix}standard deviation of \\ signal in difference image ROI\end{matrix} )}$$

Imaging analysis of the in-vivo studies was performed by three experienced radiologists (one senior resident, two attending both specialized in interventional radiology). The representation of kidneys in scans acquired before embolization was compared with those acquired after embolization. A visual analysis of the likelihood of embolization particles presence in all three modalities using a three point scale (1: particles not present, 2: particles probably present, 3: particles definitively present) was performed. Focal changes, being hyperdense in CT, dark/hypointense in MRI and dense in X-ray (in comparison to kidney parenchyma) were attributed to residing embolization particles. Since the modalities do not only image the kidney, the remaining organs within the field-of-view were also screened for signal changes through embolization.

Likelihoods of embolization effects being present were assessed for macroscopy (1: no changes, 2: probably color changes, 3: color changes definitively present) as well as histology (thrombus, residing particles, (1: no changes, 2: probably thrombus/particles visible, 3: thrombus/particles definitively present)) by a veterinarian with several years of experience in kidney research.

For data analysis sum scores over all raters, imaging modalities were calculated and compared.

### Results

### In Vitro Imaging Characterization

Within all three imaging modalities, the particles provided a clear contrast to the surrounding agar (FIG. 9). On CT and MRI, signal from particles was only found in slices representing the agar layer the particles were embedded in. Spatial distribution of signal changes matched in all three imaging modalities as seen in FIG. 9.

In CT, single particles showed maximal CT values of 206±30 HU, the density within clusters of particles was 1340±136 HU. The SNR of a single particle was 13±2.5. SNR of particle clusters in CT was 105±11.8. SNR in MR was 35±10.

### Embolization

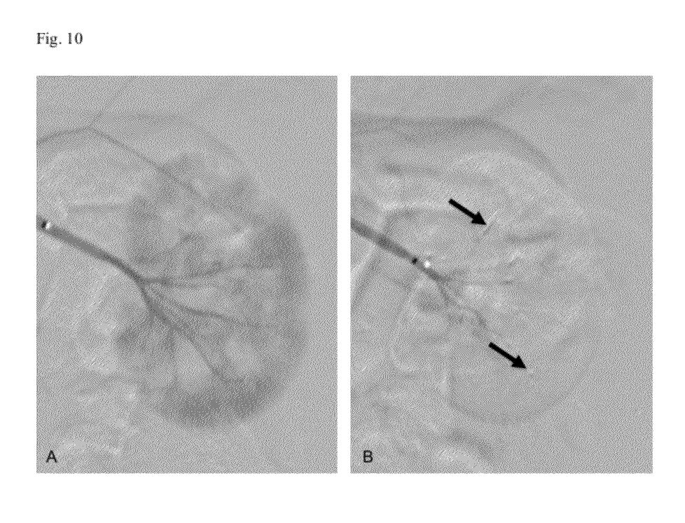

In all cases renal arteries could be successfully catheterized as confirmed by contrast media injection (FIG. 10 a). The embolization procedure could be visually observed by the performing radiologist without adding radio-opaque agents. Embolization particle injection was successful and at first without relevant resistance, yet resistance and manual injection pressure increased during the application process. The embolization was confirmed by patchy (n=2) to complete perfusion defects (n=4) (FIG. 10 b) in the post-embolization kidneys.

Image Comparison Before and after Embolization

Imaging before, after and during embolization of six rabbits was mostly successful as described in Tab. 2. In only one case X-ray angiography imaging after embolization could not be accomplished due to death of the animal after embolization due to anesthesia complications. Image comparison between scans that were performed before and after injection of embolization revealed differences that were attributed to the embolization particles:

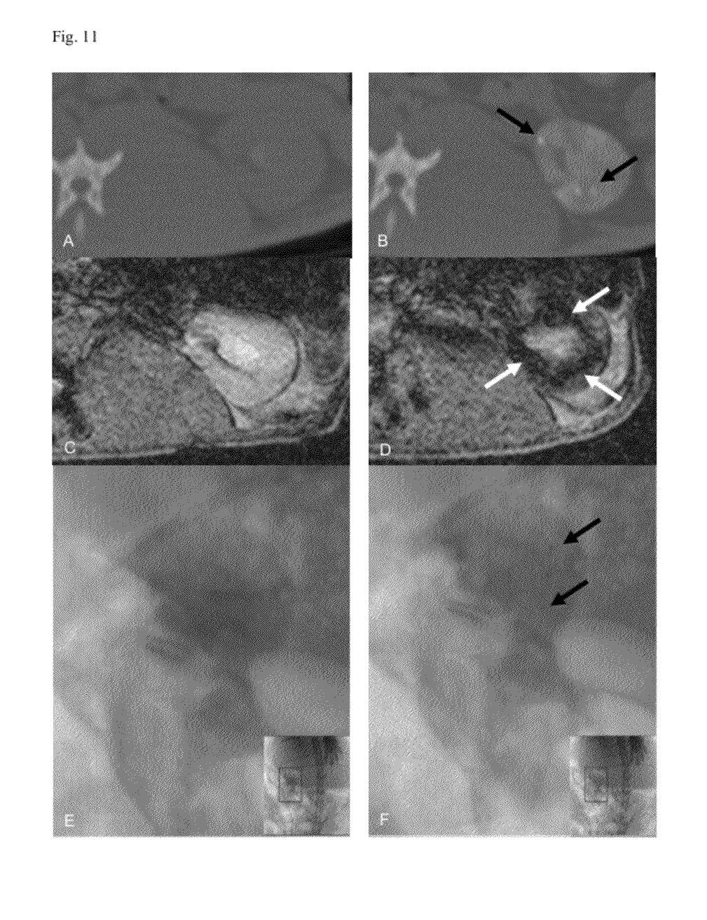

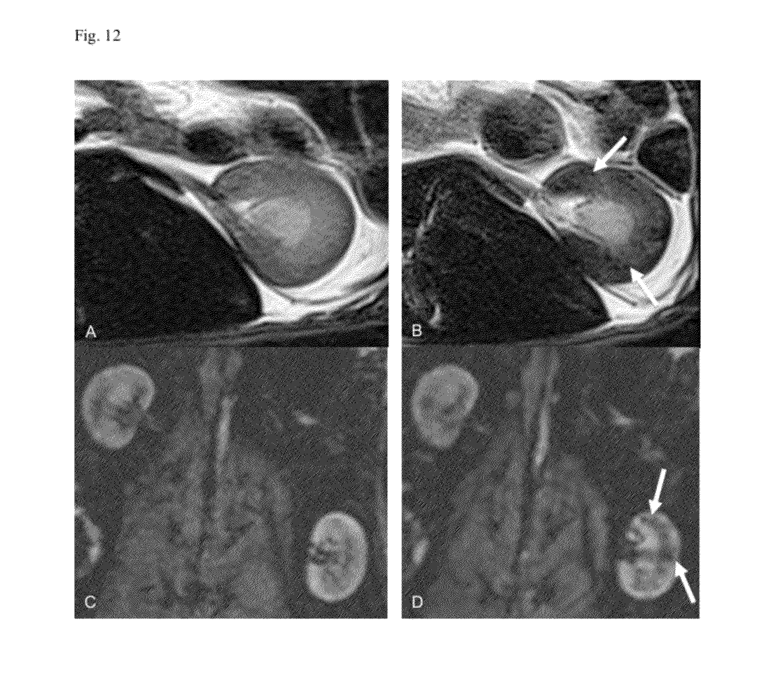

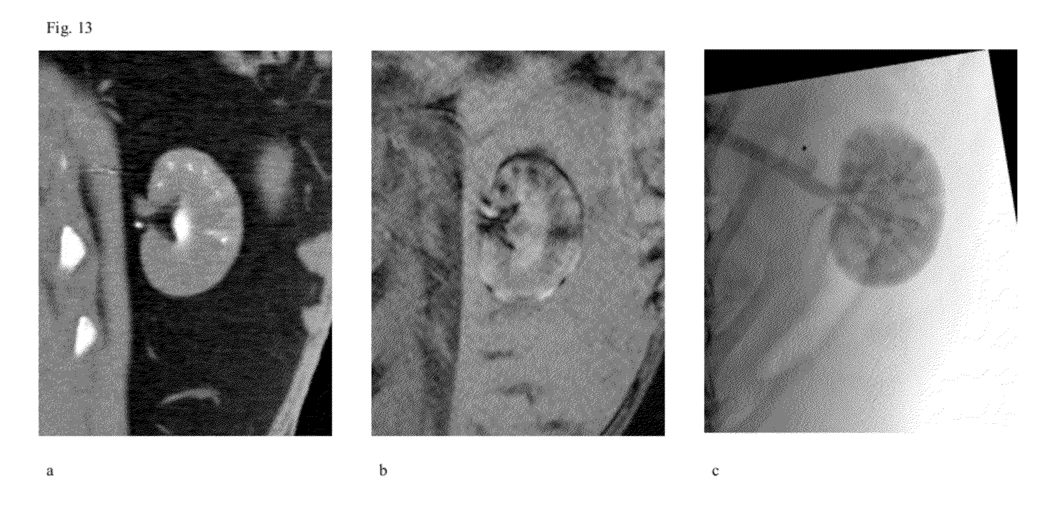

In all six cases, similar results were found: Within CT there were hyperdense, focal density changes present that were not visible in the kidney parenchyma before embolization (FIG. 11 a, b). Within MRI patchy, dark/hypointense, confluent areas in T2* weighting were visible that were not visible within kidneys before embolization (FIG. 11 c, d). Additional MR sequences also demonstrated signal changes: small focal signal drops in the kidney parenchyma in T2 weighted images (FIG. 12 a, b) as well as bigger, round hypointense parenchymal areas in the EPI sequence (FIG. 12 c, d). X-ray angiography showed focal, small hyperdense areas that were not visible in kidneys before embolization (FIG. 11 e, f). The distribution of the particles was random within kidney parenchyma and varied between animals, in some cases almost all parts of the kidney showed signal changes (n=4), whereof in other cases only parts of the kidney parenchyma showed signal changes (n=2). The distribution of signal changes in all modalities corresponded well in all kidneys (FIG. 13). Here, the embolization particles were mainly localized within the middle level and upper pole of the organ. The position of particles visibly correlated well in all three modalities.

For all raters maximum scores were reached in all cases in all imaging modalities with regard to particles being visible (resulting in an overall sum score of 153/153 for particle visibility as compared to 0/162 before embolization).

In only one case (rabbit number 5) a signal change was found that was not within kidney, here a hyper dense spot on CT and corresponding signal void in MRI was detected within the psoas muscle on the same side as the embolized kidney, which was interpreted as a misplaced embolization particle, probably due to reflux along the catheter. Beside this, no relevant signal changes from embolization or organ alterations outside the kidneys have been found.

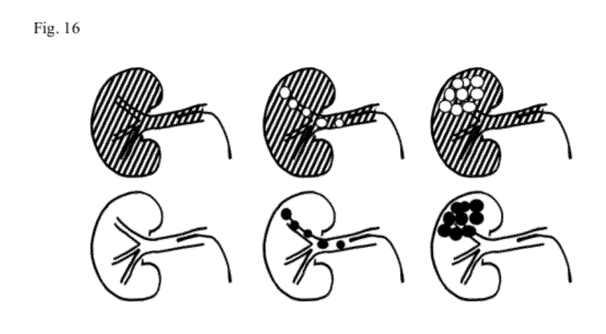

### Dynamic Imaging During Embolization (FIG. 16)

Imaging during embolization was possible in all cases, respectively twice using MRI, CT and X-ray angiography. The wash in of particles was monitored by repetitive, coronal echoplanar imaging. Here, continuously more and more dark areas caused by the susceptibility of the particles became visible. Similarly, continuously acquired CT scans showed an increasing amount of more and more hyperdense points in kidney medulla. Within angiography the particles became consecutively visible within the renal parenchyma.

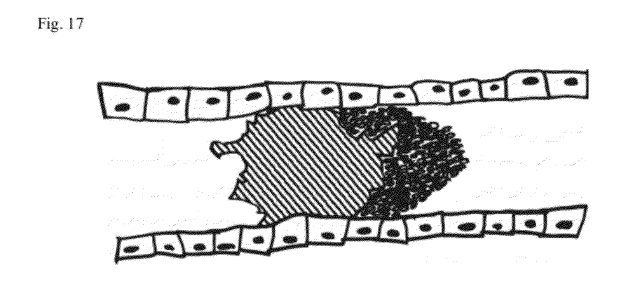

Macroscopy and Histology after Embolization (FIG. 17)

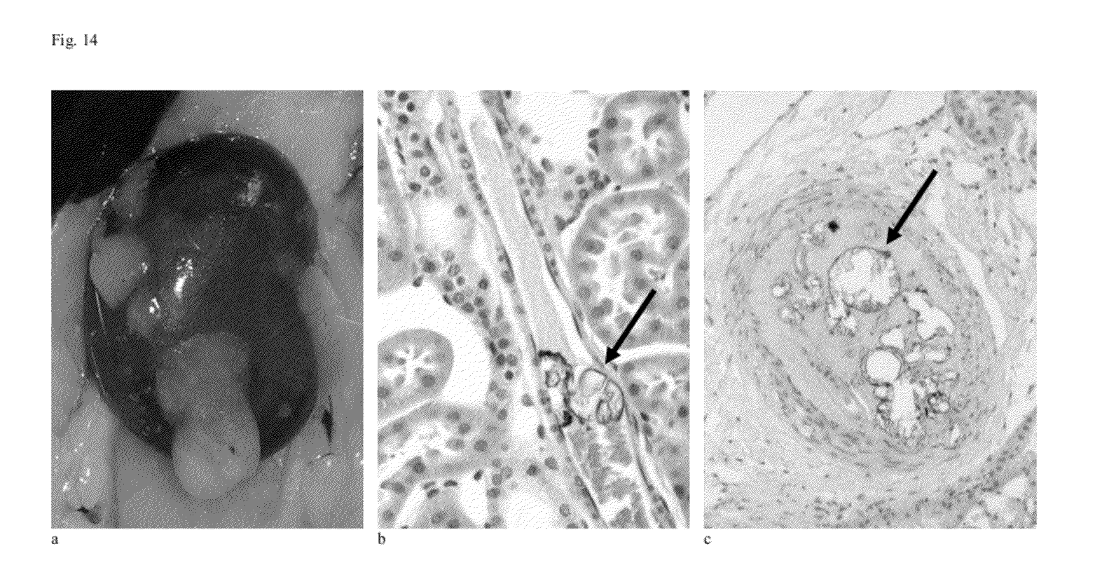

Embolized kidneys showed an inhomogeneous surface whereof some areas were darker and some brighter. The transition was sharply delineated (FIG. 14 a). Histology revealed particle inside arteries at various sites in all embolized kidneys and particles were found within interlobar, arcuate and up to the interlobular arteries. Additionally thrombi consisting of erythrocytes and fibrin were found in all vessel regions including medullary vessels (FIG. 14 b,c). No particles were found in control kidneys as well as in lung, liver and brain. The sum scores for likelihood of embolization effect was 18/18 for macroscopy and 18/18 for microscopy.

For the first time embolization particles that are visible in more than one imaging modality have been tested in-vitro an in-vivo. The results show that embolization particles according to the invention demonstrate sufficient contrast in CT, MRI and angiography so that visibility during and after application can be assured. Moreover, it could be shown that stationary tissue-residing particles are visible consistently in three imaging modalities together with histological findings of associated thrombosis.

TABLE 3
Overview of used imaging systems, scanners and employed scan parameters

| | | Scanner | Imaging | |
|---|---|---|---|---|
| No. | Modality | system | sequence | Scan parameters |
| 1 | Angiography | Integris | Abdomen | Automatic kV and mA settings (standard Abdomen DSA program, 3 frames/s), Manual |
| | | Allura, | | injection of 0.5 ml ml contrast medium for renal angiograms |
| | | (Philips, | | |
| | | Netherlands) | | |
| 2 | CT | Siemens | Static | High-resolution dual source CT scan protocol; 210 mA tube current; 120 kV tube |
| | | Definition | | voltage; 0.3 mm single slice collimation, 0.85 spiral pitch factor; 10 cm2 reconstruction |
| | | (Siemens | | field of view; H40 reconstruction kernel; 0.4 mm reconstruction increment |
| 3 | | Healthcare | Dynamic | Continuous scanning dual-source CT protocol; 305 mA tube current; 120 kV tube |
| | | Section, | | voltage; 0.3 s rotation time; 40 s total scan time; B50 reconstruction kernel. Five seconds |
| | | Germany) | | after initiating the scan, particles were slowly injected over 15 s followed by 1 ml of |
| | | | | saline |
| 4 | MRI | Magnetom | T2 | standard body coil; 2980 ms repetition time; 112 ms echo time; 140° flip angle; 1.65 mm |
| | | Tim Trio | | slice thickness; 0.2 × 0.2 mm pixel size; axial slice orientation |
| 5 | | MRI, | T2* | standard body coil, 720 ms repetition time; 20 ms echo time; flip angle 20°; slice |
| | | (Siemens | | thickness 3 mm; 0.4 × 0.4 mm pixel size; axial slice orientation |
| 6 | | Healthcare | T2 Blade | standard body coil; 3350 ms repetition time; 110 ms echo time; 140° flip angle; 3 mm |
| | | Section, | | slice thickness; 0.5 × 0.5 mm pixel size; axial slice orientation |
| 7 | | Germany, | T1 VIBE | standard body coil; fat saturation, 5 ms repetition time; 2 ms echo time; 12.5° flip angle; |
| | | 3T clinical | | 2 mm slice thickness; 0.5 × 0.5 mm pixel size; axial slice orientation |
| 8 | | scanner) | continuous | standard body coil; 1330 ms repetition time; 32 ms echo time; 90° flip angle; 2 mm slice |
| | | | EPI | thickness; 1.6 × 1.6 mm pixel size; scan duration 400 s; coronal slice orientation |

TABLE 4
Overview of animals, embolized kidneys as well as performed imaging studies and references to images

| | | | | | Imaging before | | | Dynamic imaging | | | Imaging after | | |
|---|---|---|---|---|---|---|---|---|---|---|---|---|---|
| | | Survival time after | Embolized | | embolization | | | during embolization | | | embolization | | |
| No | Weight (g) | embolization (min) | kidney | Figure | CT | MRI | X-ray | CT | MRI | X-ray | CT | MRI | X-ray |
| 1 | 2200 | 25 | right | 13a | X | X | X | | X | | X | X | X |
| 2 | 5230 | 65 | left | 11c + d, 12 | X | X | X | | X | | X | X | X |
| 3 | 2760 | 110 | right | | X | X | X | X | | | X | X | X |
| 4 | 4185 | 100 | left | | X | X | X | X | | | X | X | |
| 5 | 2920 | 80 | right | 10 e + f, | X | X | X | | | X | X | X | X |
| | | | | 13 b + c | | | | | | | | | |
| 6 | 3110 | 80 | left | 9, 10 a-d, | X | X | X | | | X | X | X | X |
| | | | | 11 a + b | | | | | | | | | |

### EXAMPLE 4

### Synthesis of Embolization Material Detectable Via CT and SPECT

The iodinated monomer MAOETIB was synthesized. Briefly, 2,3,5-triiodobenzoic acid (49 g, 0.10 mol with 0.01%) and 2,3,5-triiodobenzoic acid (1 g, 0.10 mol with 50% Iodine-131), HEMA (15 g, 0.11 mol), DCC (23 g, 0.11 mol) and 4-pyrrilidinopyridine (1.5 g, 0.010 mol) were dispersed in ether (500 ml), and then stirred at room temperature for 18 h. The formed solid was filtered off and the residue washed with fresh ether. The ether solution was then washed with HCl (2 N) and saturated NaHCO₃. The organic phase was dried over MgSO₄, filtered, and evaporated to produce an orange solid. Pure white crystals of MAOETIB (m.p. 95° C.) were obtained by the two-fold recrystallization of the orange solid from ethyl acetate (yield 84%).

The particles have been injected into living rats via a catheterization of the left kidney artery, whereof the kidney represents an accepted animal tumor embolization model. CT imaging revealed punctual signal changes within kidney parenchyma. SPECT imaging using a gamma-camera revealed high gamma radiation signal changes in the corresponding kidney areas. Histology confirmed successful embolization of vessels.

### EXAMPLE 5

### Trimodality Embolization Particles Visible Via CT, MRI, and US

Trimodality particles for embolization purposes were prepared by binding physically or covalently microbubble particles (US imaging particles) to the surface of dimodality particles.

### Synthesis of Albumin/γ-Fe₂O₃/P(MAOETIB-GMA) Trimodality Particles

The albumin/γ-Fe₂O₃/P(MAOETIB-GMA) trimodality particles were prepared according to the following steps:

### 5.1 Synthesis of MAOETIB

The iodinated monomer MAOETIB was synthesized according to following scheme. Briefly, 2,3,5-triiodobenzoic acid (50 g, 0.10 mol), HEMA (15 g, 0.11 mol), DCC (23 g, 0.11 mol) and 4-pyrrilidinopyridine (1.5 g, 0.010 mol) were dispersed in ether (500 ml), and then stirred at room temperature for 18 h. The formed solid was filtered off and the residue washed with fresh ether. The ether solution was then washed with HCl (2 N) and saturated NaHCO₃. The organic phase was dried over MgSO₄, filtered, and evaporated to produce an orange solid. Pure white crystals of MAOETIB (m.p. 95° C.) were obtained by the two-fold recrystallization of the orange solid from ethyl acetate (yield 84%).

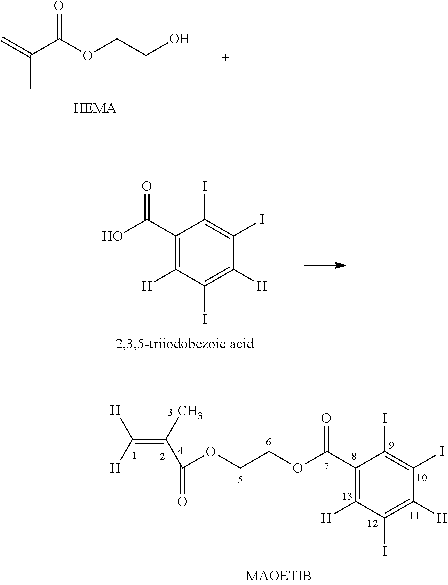

### Scheme Illustrating the Synthesis of the Monomer MAOETIB

5.2 Synthesis of the P(MAOETIB-GMA) core microparticles

P(MAOETIB-GMA) copolymeric microparticles were prepared by suspension polymerization of MAOETIB and GMA according to reference 1. Briefly, 10 ml of toluene solution containing 495 mg MAOETIB, 5 mg GMA and 40 mg BP (8% w/wₘₒₙₒₘₑᵣ) were introduced into a flask containing 100 ml of 1% PVP aqueous solution. The mixture was then stirred at 80° C. for 15 h. The organic phase containing the toluene and excess monomer was then extracted from the aqueous phase. The formed P(MAOETIB-GMA) microparticles were then washed by extensive centrifugation cycles with water and then dried by lyophilization. The dried microparticles were then sieved in fractions of sizes ranging between 40-200 μm.

### 5.3 Synthesis of the γ-Fe₂O₃/P(MAOETIB-GMA) Core-Shell Dimodality Microparticles

Magnetic γ-Fe₂O₃/P(MAOETIB-GMA) core-shell microparticles were prepared by coating the P(MAOETIB-GMA) microparticles with successive layers of γ-Fe₂O₃ nanoparticles according to reference 1. Briefly, an aqueous suspension containing 300 mg of the P(MAOETIB-GMA) microparticles in 300 ml of distilled water was mechanically stirred at 60° C. Nitrogen was bubbled through the microparticles aqueous suspension during the coating process to exclude air. Volumes of 0.5 ml of reference aqueous solutions of FeCl₂.4H₂O (25 mM) and 0.5 ml of NaNO₂ (1.5 mM) were successively introduced into the reaction flask. Then, an aqueous solution of sodium hydroxide (50 mM) was added until a pH of about 9.5 was reached. The mixture was then stirred for 1 h. This procedure was repeated 10 times. During this coating process, the surface of the P(MAOETIB-GMA) microparticles became brown-black in color. Gelatin (30 mg) was then added to the stirred aqueous suspension of the γ-Fe₂O₃/P(MAOETIB-GMA) microparticles. 30 min. later the suspension was cooled to room temperature under nitrogen atmosphere. The produced gelatin coated γ-Fe₂O₃/P(MAOETIB-GMA) microparticles were washed extensively in water and then dried by lyophilization.

### 5.4 Synthesis of Albumin Bubbles/γ-Fe₂O₃/P(MAOETIB-GMA) Trimodality Particles

Albunex (Molecular Biosystems Inc, San Diego, USA and Nycomed Imaging AS, Oslo, Norway) was bonded to the gelatin coated γ-Fe₂O₃/P(MAOETIB-GMA) particles via the carbodiimide activation method, according to the literature (3). In a typical experiment, 123 mg NHS [N-hydroxysuccinimide ester] and 82 mg CDC [1-cyclohexyl-3-(2-morpholinoethyl) carbodiimide metho-p-toluenesulfonate] were added to 15 ml MES buffer (4-morpholinolineethane sulfonic acid monohydrate, 0.1M at pH 5.0) containing 150 mg γ-Fe₂O₃/P(MAOETIB-GMA) particles. The mixture was then shaken at room temperature for 6 h. The activated particles were then washed extensively with PBS. PBS dispersion containing 20 mg albunex hollow particles was then added to 15 ml of the washed activated γ-Fe₂O₃/P(MAOETIB-GMA) particles PBS suspension. The mixture was then shaken at room temperature for additional 6 h. Unbound albunex hollow particles were then removed from the obtained albumin bubbles/γ-Fe₂O₃/P(MAOETIB-GMA) trimodality particles.

### EXAMPLE 6

### Preparation of Lipiodol-Gadolinium-Loaded PVA Microspheres

Commercially available LC Bead PVA hydrogel microspheres (100-300 μm; Biocompatibles UK, Franham, United Kingdom) were lyophilized in the presence of an excipient and then mixed with 1 mL of Lipiodol and 1.5 mL of Gd-DOTA. The loaded microspheres were rinsed with saline solution ten times. The microspheres were then dried with absorbent paper and completely vacuum-dried overnight at 40° C.

The loaded microspheres have been injected into living rabbits via a catheterization of the left kidney artery, whereof the kidney represents an accepted animal tumor embolization model. X-ray imaging revealed punctual signal changes within kidney parenchyma. MR imaging showed corresponding signal increase in T1 weighted sequences. Histology confirmed successful embolization of vessels.

In a variation the microspheres have been loaded with other paramagnetic iron oxides or other MRI contrast media in different complexes.

1. Embolization material for therapeutic use, wherein said material is visible via more than one imaging technique.

2. The embolization material of claim 1, wherein said material comprises at least one polymer component and at least one inorganic component, and wherein said material is visible with high contrast via more than one imaging technique.

3. The embolization material of claim 2, wherein said material is visible via at least one of the following imaging techniques:
a) X-ray computed tomography (CT)/projectional radiography and magnetic resonance imaging (MRI),
b) X-ray computed tomography (CT)/projectional radiography and ultrasonography (US),
c) X-ray computed tomography (CT)/projectional radiography and single photon emission computed tomography (SPECT),
d) X-ray computed tomography (CT)/projectional radiography and positron emission tomography (PET),
e) magnetic resonance imaging (MRI) and ultrasonography (US),
f) magnetic resonance imaging (MRI) and single photon emission computed tomography (SPECT),
g) magnetic resonance imaging (MRI) and positron emission tomography (PET),
h) X-ray computed tomography (CT)/projectional radiography and magnetic particle imaging, or
i) a combination of two or more of said imaging techniques.

4. The embolization material of claim 3, wherein said material is visible via three different imaging techniques at the same time.

5. The embolization material of claim 2, wherein the at least one polymer component is selected from the group of polyacrylate, polymethacrylate, polyacrylamide, polymethacrylamide, acrylate polymer, polyamide, polysiloxane, polyester, polyurethane, polyvinyl ether, polyvinyl ester, copolymers comprising as monomers a (meth)acrylic-derivative and/or a meth(acrylamide)-derivative carrying a cleavable iodine substituted side group, or mixtures thereof.

6. The embolization material according to claim 5, wherein the at least one polymer component comprises a copolymer of glycidyl-methacrylate and a (meth)acrylic-derivative carrying a cleavable iodine substituted aromatic side group.

7. The embolization material of claim 5, wherein the at least one inorganic component comprises a radio-opaque element selected from the group of calcium, iron, iodine, xenon, barium, ytterbium, silver, gold, bismuth, cesium, thorium, or tungsten, and a magnetic resonance imaging (MRI) visible component selected from the group iron oxides, gadolinium, manganese based agents, or perfluorocarbons.

8. The embolization material of claim 7, said material comprising a radio-opaque element, and a magnetic resonance imaging (MRI) visible component, and additionally components enabling detection via ultrasonography (US) selected from the group of gas aggregates or gas bubbles, microbubbles, microspheres of human albumin, microparticles of galactose, perflenapent, microspheres of phospholipids, and/or sulfur hexafluoride.

9. The embolization material of claim 1, wherein said material comprises an X-ray visible, iodine containing core, and a MRI visible, ultra small paramagnetic iron oxide based coating, and wherein said material is selected from magnetic iron oxide/Poly((2-methacryloyloxyethyl-(2,3,5-triiodobenzoate))-(glycidyl-methacrylate)) particles.

10. The embolization material of claim 9, said material exhibiting different particle sizes ranging from 30 μm to 900 μm.

11. The embolization material of claim 9, said material exhibiting different particle sizes ranging from 40 μm to 200 μm.

12. The embolization material of claim 11, wherein the first imaging technique of detection is selected from X-ray computed tomography (CT)/projectional radiography, or magnetic resonance imaging (MRI).

13. The embolization material of claim 12, wherein the second imaging technique of detection is selected from ultrasonography (US) and nuclear medical imaging techniques.

14. A kit of at least two parts for the preparation of embolization material according to claim 2, the kit comprising as one part at least one polymer component and as second part at least one inorganic component.

15. A method for the preparation of the embolization material of claim 2 comprising the steps of:
a) synthesizing the at least one polymer component,
b) synthesizing the at least one inorganic component, and
c) optionally synthesizing a component detectable via ultrasonography, and
d) combining the at least one polymer component of step a with the at least one inorganic component of step b, and optionally with the component of step c, and thus, obtaining the embolization material.

16. The embolization material of claim 3, wherein the at least one polymer component is selected from the group of polyacrylate, polymethacrylate, polyacrylamide, polymethacrylamide, acrylate polymer, polyamide, polysiloxane, polyester, polyurethane, polyvinyl ether, polyvinyl ester, copolymers comprising as monomers a (meth)acrylic-derivative and/or a meth(acrylamide)-derivative carrying a cleavable iodine substituted side group, or mixtures thereof.

17. The embolization material of claim 16, wherein the at least one inorganic component comprises a radio-opaque element selected from the group of calcium, iron, iodine, xenon, barium, ytterbium, silver, gold, bismuth, cesium, thorium, or tungsten, and a magnetic resonance imaging (MRI) visible component selected from the group iron oxides, gadolinium, manganese based agents, or perfluorocarbons.

18. The embolization material of claim 17, said material comprising a radio-opaque element, and a magnetic resonance imaging (MRI) visible component, and additionally components enabling detection via ultrasonography (US) selected from the group of gas aggregates or gas bubbles, microbubbles, microspheres of human albumin, microparticles of galactose, perflenapent, microspheres of phospholipids, and/or sulfur hexafluoride.

19. The embolization material of claim 18, wherein said material comprises an X-ray visible, iodine containing core, and a MRI visible, ultra small paramagnetic iron oxide based coating, and wherein said material is selected from magnetic iron oxide/Poly((2-methacryloyloxyethyl-(2,3,5-triiodobenzoate))-(glycidyl-methacrylate)) particles.

20. The embolization material of claim 2, wherein said material comprises an X-ray visible, iodine containing core, and a MRI visible, ultra small paramagnetic iron oxide based coating, and wherein said material is selected from magnetic iron oxide/Poly((2-methacryloyloxyethyl-(2,3,5-triiodobenzoate))-(glycidyl-methacrylate)) particles.
